(19) United States
(12) Patent Application Publication
(10) Pub. No.: US 2025/0243189 A1
(43) Pub. Date: Jul. 31, 2025

(54) HERBICIDAL IMIDAZOLE-CONTAINING COMPOUNDS

(71) Applicant: SYNGENTA CROP PROTECTION AG, Basel (CH)

(72) Inventors: Suzanna Jane DALE, Bracknell, Berkshire (GB); Philip Michael ELVES, Bracknell, Berkshire (GB); Charles William Frederick KINGSTON, Bracknell, Berkshire (GB); James Alan MORRIS, Bracknell, Berkshire (GB); Vikas SIKERVAR, Bracknell, Berkshire (GB); Samuel Vaughan WATKIN, Bracknell, Berkshire (GB)

(73) Assignee: SYNGENTA CROP PROTECTION AG, Basel (CH)

(21) Appl. No.: 18/878,521

(22) PCT Filed: Jun. 16, 2023
(86) PCT No.: PCT/EP2023/066268

§ 371 Date: Dec. 23, 2024

(30) Foreign Application Priority Data
Jun. 24, 2022 (GB) 2209286.0

(51) Int. Cl. C07D 409/14; A01N 43/54; A01N 43/56; A01N 43/76; A01N 43/78; A01N 43/80; A01N 43/82; A01P 13/02; C07D 403/14; C07D 405/14; C07D 413/14; C07D 417/14

(52) U.S. Cl. CPC C07D 409/14; A01N 43/54; A01N 43/56; A01N 43/76; A01N 43/78; A01N 43/80; A01N 43/82; A01P 13/02; C07D 403/14; C07D 405/14; C07D 413/14; C07D 417/14

(57) ABSTRACT

The present invention relates to compounds of Formula (I) or an agronomically acceptable salt of said compounds wherein A, Q, R¹, R², R⁴ and m are as defined herein. The invention further relates to herbicidal compositions which comprise a compound of Formula (I) and to the use of compounds of Formula (I) for controlling weeds, in particular in crops of useful plants.

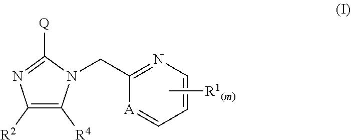

The present invention relates to herbicidal compounds, to processes for their preparation, to herbicidal compositions which comprise the novel compounds, and to their use for controlling weeds, in particular in crops of useful plants, or for inhibiting plant growth.

WO2022/101270 discloses herbicidal N-heteroaryl pyrazole compounds. WO2023/066783 discloses herbicidal imidazole compounds which feature a phenyl or 6-membered heteroaryl in position Q identified below.

Thus, according to the present invention there is provided a compound of Formula (I):

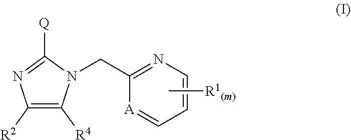

or an agronomically acceptable salt thereof,
wherein
A is CR⁵ or N;
Q is 5-membered heteroaryl which is optionally substituted by 1 or 2 R³ substituents,
R¹ is independently selected from the group consisting of halogen, —CN, C₁-C₂alkyl, C₁-C₂haloalkyl, C₃-C₆cycloalkyl, C₁-C₂alkoxy- and C₁-C₂haloalkoxy-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)ₚC₁-C₄alkyl, —C(R⁶)═NOR⁷ and C₃-C₆cycloalkyl;
R³ is independently selected from the group consisting of halogen, C₁-C₄ alkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁴ is independently selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁵ is selected from the group consisting of hydrogen, fluoro, chloro and —CN;
R⁶ is hydrogen or C₁-C₄alkyl;
R⁷ is hydrogen or C₁-C₂alkyl;
R⁸ is hydrogen or C₁-C₄alkyl;
R⁹ is hydrogen or C₁-C₄alkyl;
m=1 or 2; and
p=0, 1 or 2.

C₁-C₄alkyl- and C₁-C₆alkyl- includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine. The same correspondingly applies to halogen in the context of other definitions, such as haloalkyl.

C₁-C₆haloalkyl- includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl. C₁-C₄haloalkyl- and C₁-C₂haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄alkoxy and C₁-C₂alkoxy includes, for example, methoxy and ethoxy.

C₁-C₆haloalkoxy- and C₁-C₄haloalkoxy- include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₄alkenyl- includes, for example, —CH═CH₂ (vinyl) and —CH₂—CH═CH₂ (allyl).

C₂-C₄alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

C₁-C₄alkyl-S- (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)- (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂- (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention, A is N. In another embodiment of the present invention m is 1. Thus, in a preferred embodiment of the present invention there is provided a compound of Formula (Ia)

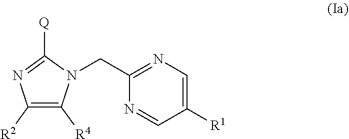

wherein Q, R¹, R² and R⁴ are as defined in claim 1 above.

In one embodiment of the present invention, R¹ is preferably chloro. In another embodiment of the present invention, R⁴ is preferably hydrogen. In another embodiment of the present invention, R² is preferably —CF₃ or —CF₂H.

In another embodiment of the present invention, Q is preferably selected from the group consisting of:

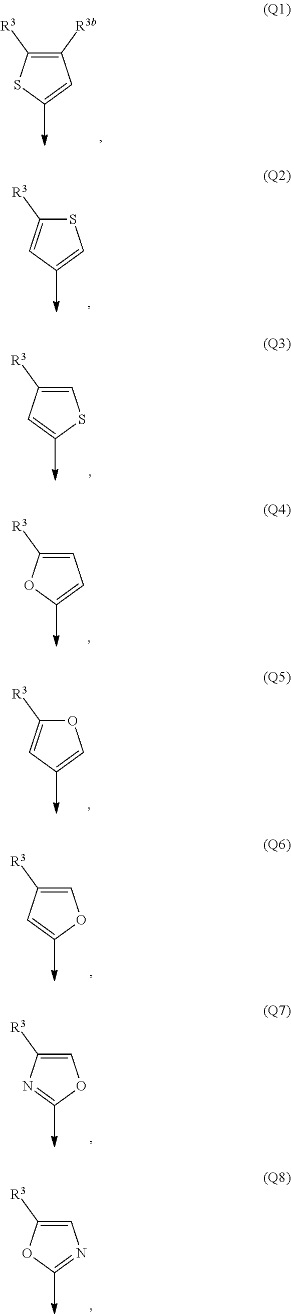

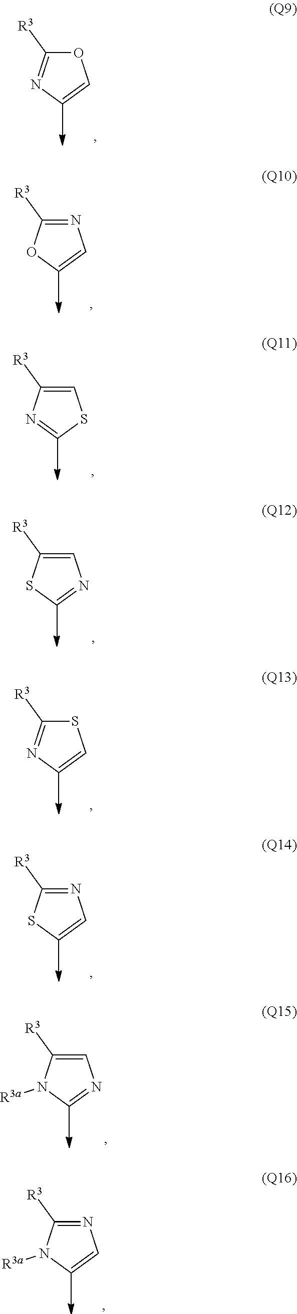

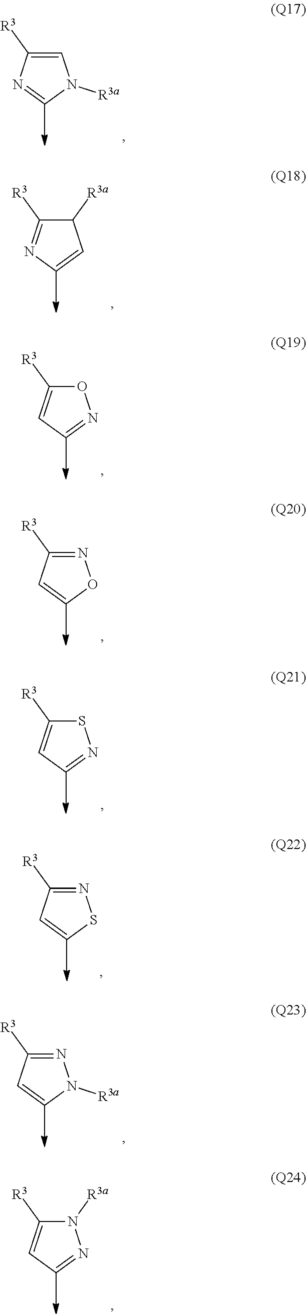

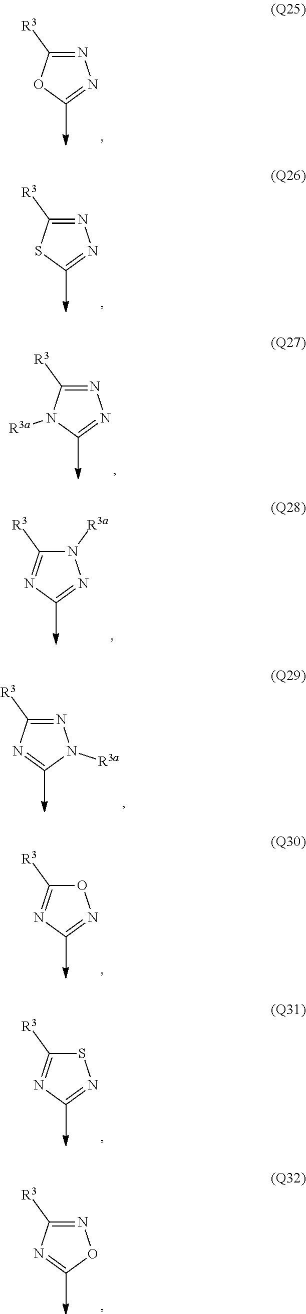

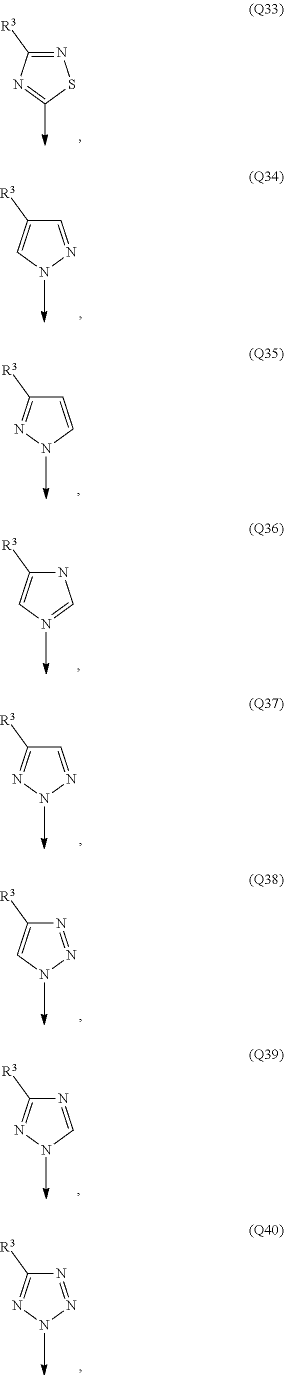

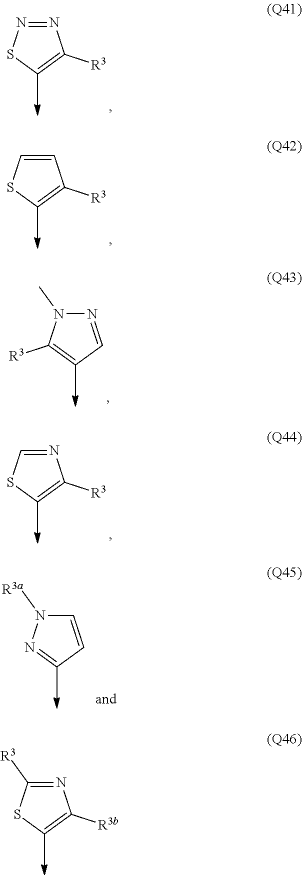

wherein R³ is hydrogen or defined in claim 1 above, R^{3a} is selected from the group consisting of hydrogen, methyl, ethyl, CHF₂ and cyclopropyl and R^{3b} is selected from the group consisting of hydrogen, fluoro, chloro and bromo.

In a more preferred embodiment Q is selected from the group consisting of Q-1, Q-2, Q-3, Q-4, Q-13, Q-14, Q-15, Q-16, Q-22, Q-23, Q-24, Q-41, Q-42 and Q-43. In a more preferred embodiment Q is selected from the group consisting of Q-1, Q-3, Q-4, Q-13, Q-14, Q-15, Q-16, Q-23, Q-24, Q-41, Q-42 and Q-43. In an even more preferred embodiment, Q is selected from the group consisting of Q-1, Q-14, Q-22, Q-23, Q-41 and Q-43.

In one embodiment of the present invention, R³ is chloro.

The present invention further relates to compounds of Formula VIIIa:

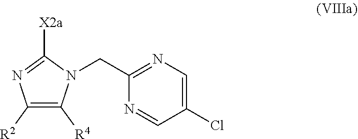

wherein R² and R⁴ are as defined above and X2a is hydrogen or halogen (preferably bromo or iodo). In a preferred embodiment, R² is —C(O)OC₁-C₄alkyl (e.g —C(O)OC₂H₅), C₁-C₄haloalkyl (e.g —CHF₂ or —CF₃) and R⁴ is hydrogen.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, broclozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), fluchloraminopyr (including fluchloramino-tefuryl), flufenacet, flufenoximacil, flumetsulam, flumioxazin, fluometuron, fomesafen flupyrsulfuron (including flupyrsulfuron-methyl-sodium), fluroxypyr (including fluroxypyr-meptyl), flusulfinam, fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, icafolin (including icafolin-methyl), imazamox (including R-imazamox), imazapic, imazapyr, imazethapyr, indaziflam, indolauxipyr (including indolauxipyr-cyanomethyl), iodosulfuron (including iodosulfuron-methyl-sodium), iofensulfuron (including iofensulfuron-sodium), ioxynil, iptriazopyrid, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyraquinate, pyrasulfotole, pyridate, pyriftalid, pyriflubenzoxim, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, (1RS,5SR)-3-[2-methoxy-4-(prop-1-yn-1-yl)phenyl]-4-oxobicyclo[3.2.1]oct-2-en-2-yl methyl carbonate, ethyl-2-[[3-[[3-chloro-5-fluoro-6-[3-methyl-2,6-dioxo-4-(trifluoromethyl)pyrimidin-1-yl]-2-pyridyl]oxy]acetate, methyl 2-[2-[2-bromo-4-fluoro-5-[3-methyl-2,6-dioxo-4-(trifluoromethyl)pyrimidin-1-yl]phenoxy]phenoxy]-2-methoxy-acetate, 6-chloro-4-(2,7-dimethyl-1-naphthyl)-5-hydroxy-2-methyl-pyridazin-3-one, (2-fluorophenyl)methyl 6-amino-5-chloro-2-(4-chloro-2-fluoro-3-methoxy-phenyl)pyrimidine-4-carboxylate, 6-amino-5-chloro-2-(4-chloro-2-fluoro-3-methoxy-phenyl)pyrimidine-4-carboxylic acid, and methyl 3-[2-chloro-5-[3,6-dihydro-3-methyl-2,6-dioxo-4-(trifluoromethyl)-1(2H)-pyrimidinyl]-4-fluorophenyl]-3a,4,5,6-tetrahydro-6-methyl-6aH-cyclopent [d] isoxazole-6a-carboxylate.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention may also be used in conjunction with plants disclosed in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

### Processes for Preparation of Compounds of Formula (I)

Processes for preparation of compounds, e.g. a compound of formula (I) (which optionally can be an agrochemically acceptable salt thereof), are now described, and form further aspects of the present invention.

A compound of formula I-3 is a compound of Formula I, wherein Q is a C-linked 5-membered heterocycle, R², A and R¹₍ₘ₎ are as defined in formula I, and in which R₄₁ is C₁-C₄alkyl and may be prepared by a Suzuki reaction, which involves for example, reacting compounds of formula I-1,

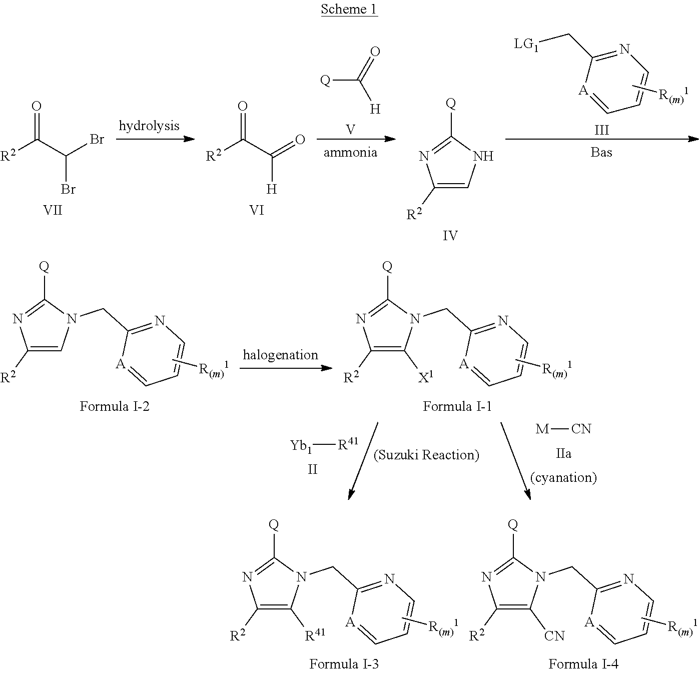

wherein Q is a C-linked 5-membered heterocycle, R₂, A and R¹₍ₘ₎ are as defined in formula I, and wherein X¹ is chlorine, bromine or iodine, with compounds of formula II, wherein R₄₁ is C₁-C₄alkyl, and wherein Yb₁ can be a boron-derived functional group, such as for example B(OH)₂ or B(ORb₁)₂ wherein Rb₁ can be a C₁-C₄alkyl group or the two groups ORb₁ can form together with the boron atom a five membered ring, as for example a pinacol boronic ester. The reaction may be catalyzed by a palladium based catalyst, for example tetrakis (triphenyl-phosphine) palladium(0), (1,1′bis(diphenylphosphino)ferrocene)dichloro-palladium-dichloromethane (1:1 complex) or chloro (2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example cyclopentyl methyl ether, dioxane, acetonitrile, N,N-dimethyl-formamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art.

A compound of Formula I-4 is a compound of Formula I where R⁴=CN and may be prepared from compounds of formula I-1, by reaction with M-CN IIa (cyanation), wherein M is a metal coordinated to the cyanide. Examples of cyanating reagent include NaCN, Zn(CN)₂, or potassium ferrocyanide amongst others. The reaction may be catalyzed by a palladium based catalyst, for example tetrakis(triphenylphosphine)palladium(0), (1,1′bis(diphenylphosphino)ferrocene) dichloro-palladium-dichloromethane (1:1 complex) or chloro(2-dicyclohexylphosphin o-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethyl-formamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such reactions are well known to those skilled in the art.

Compounds of formula I-1, can be prepared by a halogenation reaction, which involves for example, reacting compounds of formula I-2, wherein Q is a C-linked 5-membered heterocycle, R₂, A and R¹₍ₘ₎ are as defined in formula I, with halogenating reagents such as N-chlorosuccinimide (NCS), N-bromosuccinimide (NBS), or N-iodosuccinimide (NIS), optionally in the presence of an additive, such as for example p-toluenesulfonic acid. Alternatively, the halogenation may involve chlorine, bromine or iodine. Such halogenation reactions are carried out in a suitable solvent, such as chloroform, carbon tetrachloride, 1,2-dichloroethane, acetic acid, diethyl ether, acetonitrile or N,N-dimethylformamide, at temperatures between 20-200° C., preferably room temperature to 100° C.

Compounds of formula I-2, can be prepared by reacting compounds of formula IV, wherein Q is a C-linked 5-membered heterocycle and R₂ are as defined in formula I with reagents of the formula III, wherein A and R¹₍ₘ₎ are as defined in formula I, and in which LG₁ is a halogen, preferably iodine, bromine or chlorine (or a pseudo-halogen leaving group, such as a (halo alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, N,N-dimethylacetamide or acetonitrile and the like, at temperatures between 0 and 120° C., by procedures well known to those skilled in the art.

Compounds of formula IV, can be prepared by condensation reaction of compounds of formula VI or its hydrated form, wherein R² is as defined in formula I with compounds of formula V, wherein Q is a C-linked 5-membered heterocycle in the presence of ammonia or its surrogates such as ammonium hydroxide. The reaction can be carried out in the presence of solvent such as methanol, tetrahydrofuran, ethanol, amongst others and at temperatures between 20-200° C., preferably room temperature to 100° C. Compounds of formula VI or its hydrated form, wherein R² is as defined in formula I can be prepared by hydrolysis of compounds of formula VII, wherein R² is as defined in formula I. Such two step reactions are well known in literature.

Alternatively, compounds of formula I, wherein Q, R², A, R⁴ and R¹₍ₘ₎ are as defined in formula I above can be prepared following scheme 2.

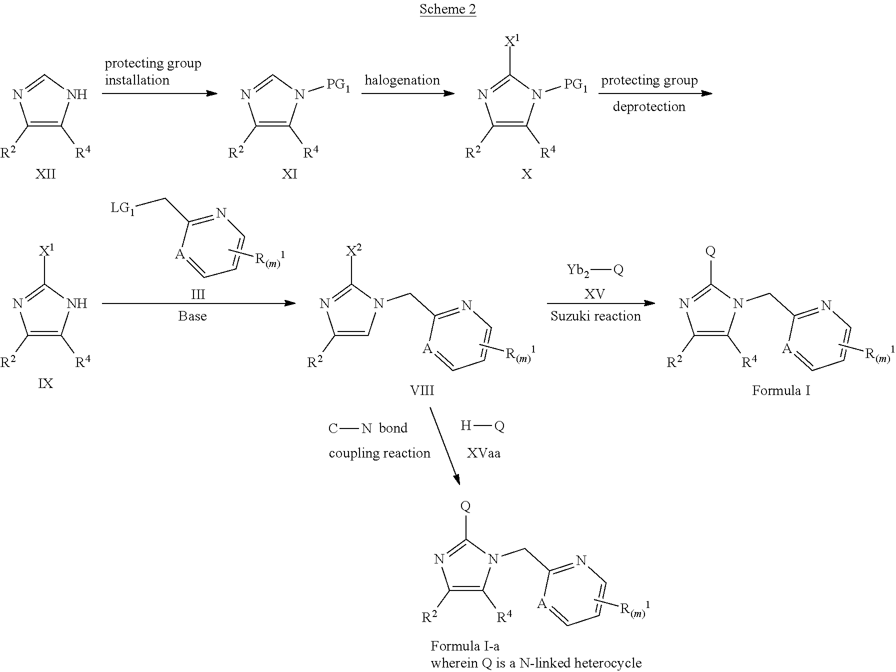

In scheme 2 compounds of formula I can be prepared by Suzuki cross-coupling reaction between compounds of formula VIII, wherein R², A, R⁴ and R¹₍ₘ₎ are as defined in formula I above and X² is a leaving group like, for example, chlorine, bromine or iodine, with compounds of formula Yb₂-Q XV wherein Q is as defined in formula I above and Yb₂ can be a boron-derived functional group, such as for example B(OH)₂ or B(OR_{b2})₂ wherein Roz can be a C₁-C₄alkyl group or the two groups OR_{b2} can form together with the boron atom a five membered ring, as for example a pinacol boronic ester following procedure analogous to as described in Scheme 1 for the conversion of compounds of formula I-1 to compounds of formula I-3.

Compounds of formula I-a wherein Q is a N-linked heterocycle can be prepared from compounds of formula VIII via C-N cross-coupling reaction. Such reactions are well known in the literature and can be carried out in the presence of a metal catalyst such as using palladium based catalyst like dichloro-(chloromethylchloronio)-bis[cyclopentyl(diphenyl)phosphaniumyl]palladium(3-); iron or copper based catalyst like tetrakis(acetonitrile)copper(I) tetrafluoroborate and optionally in the presence of a ligand such as 8-hydroxyquinoline and in the presence of a base such as potassium carbonate or potassium phosphate and in the presence of a solvent such as acetonitrile, tetrahydrofuran, dioxane, toluene, xylene and optionally in the presence of a microwave at temperature between 40° C. and 200° C. Compounds of formula VIII, wherein R², R⁴, A and R¹₍ₘ₎ are as described in formula I above and X₂ is a leaving group like, for example, chlorine, bromine or iodine, can be prepared by alkylation reaction of compounds of formula IX, wherein R², R⁴, are as described in formula I above and X² is a leaving group like, for example, chlorine, bromine or iodine with compounds of formula III, wherein A and R¹₍ₘ₎ are as described in formula I above and LG₁ is a halogen, preferably iodine, bromine or chlorine (or a pseudo-halogen leaving group, such as a (halo) alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N, N-dimethylformamide DMF, N,N-dimethylacetamide or acetonitrile and the like, at temperatures between 0 and 120° C., by procedures well known to those skilled in the art.

Compounds of formula IX can be prepared by protecting group deprotection reaction from compounds of formula X, wherein R₂, R₄ are as defined in formula I above, X² is a leaving group like, for example, chlorine, bromine or iodine and PG₁ is a N-protecting group for example acetyl, trimethylsilylethoxymethyl (SEM), tert-butyloxycarbonyl amongst others amino protecting groups. Such reactions are well known to those skilled in the art and can be carried out for example using base catalyzed or acid catalyzed such as HCl. Compounds of formula X, wherein X₂ is a leaving group like, for example, chlorine, bromine or iodine and PG₁ is a N-protecting group for example acetyl, trimethylsilylethoxymethyl (SEM), tert-butyloxycarbonyl amongst others amino protecting groups can be prepared from compounds of formula XI wherein PG₁ is a N-protecting group for example acetyl, trimethylsilylethoxymethyl (SEM), tert-butyloxycarbonyl amongst others amino protecting groups using halogenation reaction. Such reactions can be carried out in a two-step procedure which involved metalation using strong base such as butyl lithium, tert-butyl lithium, lithium tetramethylpiperidide, lithium diisopropylamide amongst others bases and quenching with suitably desired halogenating reagent such as molecular iodine, bromine or chlorine. Such reactions are well known in the literature and described for example in Journal of Organic Chemistry (1993), 58(5), 997-8. Compounds of formula XI can be prepared from compounds of formula XII by protection group installation. Such reactions can be carried out in the presence of base such as sodium hydride, potassium carbonate, sodium carbonate, and in the presence of suitable protecting group reagents such as 2-(trimethylsilyl) ethoxymethyl chloride, acetyl chloride, di-tert-butyl dicarbonate and in the presence of solvent such as tetrahydrofuran, methanol, water, acetonitrile, dimethylformamide. Such reactions are well known in the literature.

Alternatively compounds of formula I, can be prepared by metal catalyzed C—H activation and arylation by reacting compounds of formula XIII and compounds of formula XVI, wherein X³ is a halogen, preferably iodine, bromine or chlorine (scheme 3).

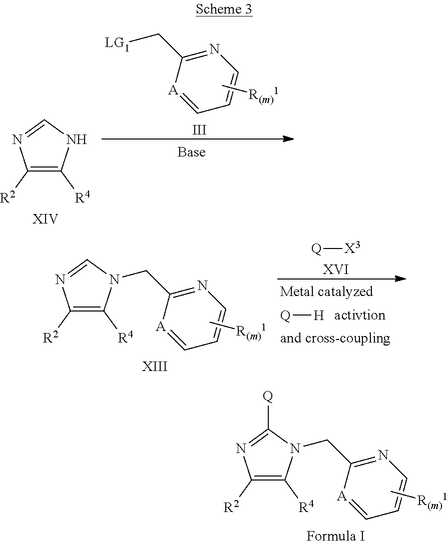

The reaction can be carried out in the presence of a palladium catalyst such as palladium acetate, bis(acetonitrile)dichloropalladium(II), tris(dibenzylideneacetone)dipalladium(0), palladium(π-cinnamyl) chloride dimer or copper catalyst such as copper iodide, copper acetate, copper bromide, copper, or combination of these palladium and copper catalysts. The reaction is generally carried out in the presence of ligand such as phosphine based ligands for example triphenylphosphine, tricyclohexylphosphine or 1,4-bis(diphenylphosphino)butane and base such as potassium carbonate, 1,8-diazabicyclo 5.4.0 undec-7-ene, potassium phosphate, potassium acetate or cesium carbonate. The reaction can be carried out optionally under microwave irradiation and at temperature in the range of 20° C. to 200° C. and in the presence of solvent such as xylene, toluene, dioxane, acetic acid or dimethylformamide. Such reactions are described in literature.

Compounds of formula XIII, wherein R² and R⁴ are as described in formula I above can be prepared by reacting compounds of formula XIV, wherein R² and R⁴ are as described in formula I above and compounds of formula III, wherein LG₁ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide, N, N-dimethylacetamide or acetonitrile and the like, at temperatures between 0 and 120° C., by procedures well known to those skilled in the art.

A compound of formula I-5 is a compound of Formula I, wherein R² is —CF₂H, R⁴ is —H and Q and R¹₍ₘ₎ are as defined in formula I. Compounds of formula I-5 can be prepared following scheme 4. In scheme 4 compounds of formula I-5 can be prepared by Suzuki cross-coupling reaction between compounds of formula XXV, wherein X³ is a leaving group like, for example, chlorine, bromine or iodine, with compounds of formula Yb₃-Q XXVI wherein Q is as defined in formula I above and Yb₃ can be a boron-derived functional group, such as for example B(OH)₂ or B(OR_{b3})₂ wherein R_{b3} can be a C₁-C₄alkyl group or the two groups OR_{b3} can form together with the boron atom a five membered ring, as for example a pinacol boronic ester following procedure as described in scheme 4 for the conversion of compounds of formula VIII to compounds of formula I-a.

Compounds of formula XXV can be prepared from compounds of formula XXIV via fluorination reactions using fluorinating reagents such as diethylaminosulfur trifluoride or bis(2-methoxyethyl)aminosulfur trifluoride amongst others. Compounds of formula XXIV can be prepared from compounds of formula XXIII via oxidation reaction using oxidizing reagents such as MnO₂, SO₃. pyridine, pyridinium dichromate or pyridinium chlorochromate amongst other alcohol oxidizing reagents.

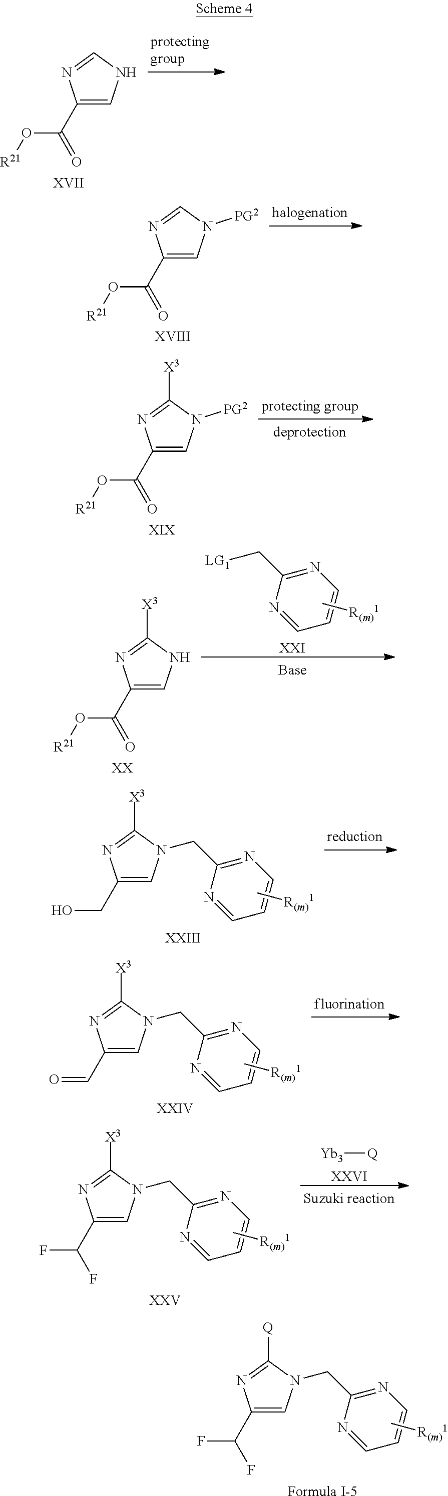

Alternatively compounds of formula XXIV can be prepared following scheme 5.

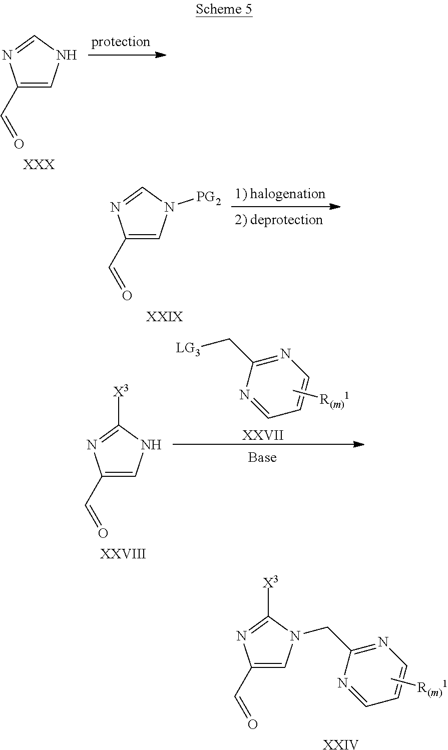

compounds of formula XXIV can be prepared by reacting compounds of formula XXVIII with compounds of formula XXVII following procedure analogous to as described in scheme 1 for the conversion of compounds of formula IV to compounds of formula I-2. Compounds of formula XXVIII can be prepared from compounds of formula XXIX via halogenation followed by N-deprotection reaction. Halogenation reactions can be carried out in one step under radical conditions using halogenating reagent such as N-bromosuccinimide in the presence of a radical initiator such as azobisisobutyronitrile. Deprotection reactions are well known to those skilled in the art and can be carried out for example using base catalysed or acid catalysed such as HCl. Compounds of formula XXIX can be prepared from compound of formula XXX by protection group installation. Such reactions can be carried out in the presence of base such as sodium hydride, potassium carbonate, sodium carbonate, and in the presence of suitable protecting group reagents such as 2-(trimethylsilyl)ethoxymethyl chloride, acetyl chloride, di-tert-butyl dicarbonate and in the presence of solvent such as tetrahydrofuran, methanol, water, acetonitrile, dimethylformamide.

Compounds of formula XXIII can be prepared from compounds of formula XXII, wherein R²¹ is C₁-C₆alkyl via reduction reactions using reducing agents such as lithium aluminium hydride or diisobutylaluminium hydride. Compounds of formula XXII can be prepared by reacting compounds of formula XX with compounds of formula XXI, wherein LG₂ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, sulfolane, N,N-dimethylacetamide or acetonitrile and the like, at temperatures between 0 and 120° C., by procedures well known to those skilled in the art. Compounds of formula XX can be prepared from compounds of formula XVII in three steps (scheme 4) following procedure analogous to as described in scheme 2 for the conversion of compounds of formula XII to compounds of formula IX.

Alternatively compounds of formula I-5 can be prepared following scheme 6.

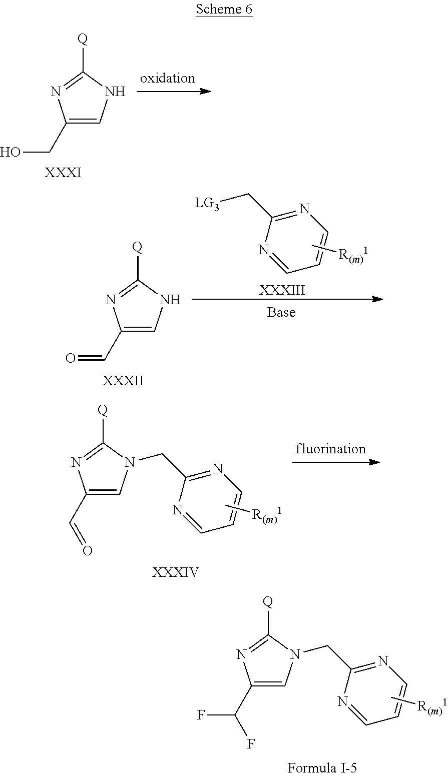

In scheme 6 compounds of formula I-5 can be prepared from compounds of formula XXXIV via fluorination reaction using fluorinating reagents such as diethylaminosulfur trifluoride or bis(2-methoxyethyl)aminosulfur trifluoride amongst others. Compounds of formula XXXIV can be prepared by reacting compounds of formula XXXII with compounds of formula XXXIII, wherein LG₃ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, sulfolane, N,N-dimethylacetamide or acetonitrile and the like, at temperatures between 0 and 120° C., by procedures well known to those skilled in the art. Compounds of formula XXXII can be prepared from compounds of formula XXXI via oxidation reaction using oxidizing agents such as MnO₂, SO₃.pyridine, pyridinium dichromate or pyridinium chlorochromate amongst other alcohol oxidizing reagents. Compounds of formula XXXI can be prepared following procedure reported in literature for example in J. Med. Chem. 1995, 38, 2251-2255.

Alternatively compounds of formula XXXIV can be prepared following scheme 7. In scheme 7, compound of formula XXXIV can be prepared by reacting compounds of formula XXXII with compounds of formula XXXIII, following procedure as described in scheme 6.

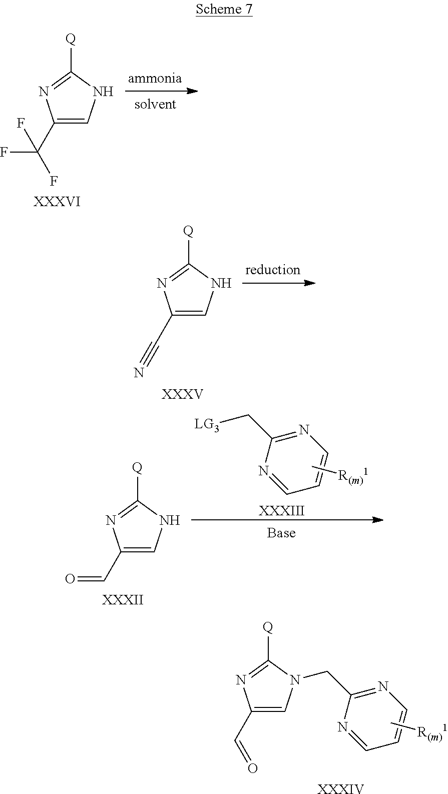

Compound of formula XXXII can be prepared by reacting compound of formula XXXV with a suitable reducing agent such as diisobutyl aluminium hydride. Compound of formula XXXV can be prepared by reacting compound of formula XXXVI with ammonium hydroxide or similar other ammonia surrogates to transform the trifluoromethyl group to a cyano group. Such reactions are well documented in the literature (see for example Matthews, D. P.; Whitten, J. P.; Mccarthy, J. R. J. Org. Chem. 1986, 51, 3228). Synthesis of imidazole compounds of Formula XXXVI are well documented in the literature (see for example Journal of Medicinal Chemistry, 2000, 43, 2165 and Synthetic Communications, 2020, 50, 700).

The following non-limiting examples provide specific synthesis methods for representative compounds of the present invention, as referred to in the Table below.

### EXAMPLE 1: PREPARATION OF 5-CHLORO-2-[[2-(5-CHLORO-2-THIENYL)-4-(TRIFLUOROMETHYL)IMIDAZOL-1-YL]METHYL]PYRIMIDINE (1.001)

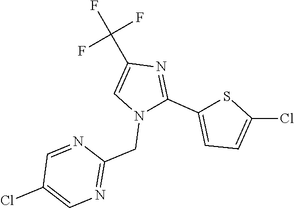 [SMILES: FC(F)(F)c1cn(Cc2ncc(Cl)cn2)c(-c2ccc(Cl)s2)n1]

### Step 1: Preparation of 3,3,3-trifluoro-2-oxo-propanal (I5)

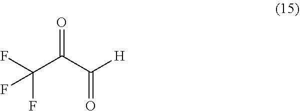

To a 100 mL flask was added sodium acetate (1.41 g, 17.05 mmol) and water (5.00 mL). 1,1-dibromo-3,3,3-trifluoroacetone (2.325 g, 8.186 mmol) was added to the above solution giving a turbid solution/suspension. The reaction mixture was then heated to 100° C. for 40 minutes. The reaction mixture was then cooled to room temperature and used as such for the next step.

### Step 2: Preparation of 2-(5-chloro-2-thienyl)-4-(trifluoromethyl)-1H-imidazole (I6)

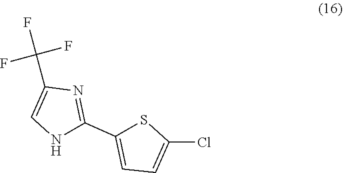 [SMILES: FC(F)(F)c1c[nH]c(-c2ccc(Cl)s2)n1]

To a 3-necked 250 mL flask was added 5-chlorothiophene-2-carbaldehyde (1.000 g, 6.822 mmol), methanol (20.00 mL) and 35% aqueous ammonia (5.00 mL, 44 mmol) giving a colourless solution. To this was added the reaction mixture of 15 prepared in step 1 above slowly via dropping funnel dropwise over 30 mins. After complete addition the reaction was stirred at room temperature for 3 hours. The organics were removed under vacuum and the residue was partitioned between ethyl acetate and water and the phases separated. The aqueous phase was extracted with ethyl acetate (3×20 mL). The organics were combined, washed with saturated brine and concentrated on to granulated celite. The crude material was subjected to column chromatography on silica gel using 0-100% ethyl acetate in cyclohexane to give 2-(5-chloro-2-thienyl)-4-(trifluoromethyl)-1H-imidazole (I6) as a pale-yellow solid (0.952 g, 55% yield). ¹H NMR (400 MHz, CD₃OD) δ=7.65-7.58 (m, 1H), 7.41-7.36 (m, 1H), 7.09-7.00 (m, 1H)

### Step 3: Preparation of 5-chloro-2-[[2-(5-chloro-2-thienyl)-4-(trifluoromethyl)imidazol-1-yl]methyl]pyrimidine (1.001)

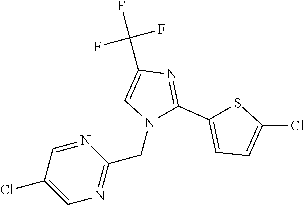 [SMILES: FC(F)(F)c1cn(Cc2ncc(Cl)cn2)c(-c2ccc(Cl)s2)n1]

To a 25 ml flask was added 2-(5-chloro-2-thienyl)-4-(trifluoromethyl)-1H-imidazole I6 (0.100 g, 0.396 mmol,) in acetonitrile (1.32 ml) and water (0.06 ml), followed by potassium carbonate (0.164 g, 1.19 mmol), 5-chloro-2-(chloromethyl)pyrimidine hydrochloride (0.0997 g, 0.475 mmol) and potassium iodide (0.0131 g, 0.0792 mmol). The reaction mixture was stirred and heated to 70° C. After 1.5 hours, the reaction mixture was allowed to cool to room temperature. The reaction mixture was diluted with water (10 mL) and brine (10 mL) and extracted with ethyl acetate (2×15 mL). The combined organics were passed through a hydrophobic frit and evaporated under reduced pressure to give the crude product. This was subjected to column chromatography on silica gel using a gradient of 0-30% ethyl acetate in cyclohexane to give 5-chloro-2-[[2-(5-chloro-2-thienyl)-4-(trifluoromethyl)imidazol-1-yl]methyl]pyrimidine 1.001 as a pale yellow gum (96 mg, 73% yield). ¹H NMR (400 MHz, CDCl₃) δ=8.71 (s, 2H), 7.47 (d, 1H), 7.23 (d, 1H), 6.89 (d, 1H), 5.45 (s, 2H)

### EXAMPLE 2: PREPARATION OF 5-CHLORO-2-[[2-(3-METHYLIMIDAZOL-4-YL)-4-(TRIFLUOROMETHYL)IMIDAZOL-1-YL]METHYL]PYRIMIDINE (1.013)

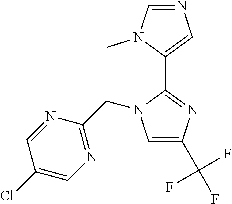 [SMILES: Cn1cncc1-c1nc(C(F)(F)F)cn1Cc1ncc(Cl)cn1]

### Step 1: Preparation of 5-chloro-2-[[4-(trifluoromethyl)imidazol-1-yl]methyl]pyrimidine (I7)

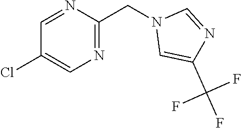 [SMILES: FC(F)(F)c1cn(Cc2ncc(Cl)cn2)cn1]

To a solution of 4-(trifluoromethyl)-1H-imidazole (204 mg, 1.47 mmol) in MeCN (4.8 mL) and H₂O (0.04 mL) was added K₂CO₃ (603 mg, 4.3629 mmol) and 5-chloro-2-(chloromethyl)pyrimidine hydrochloride (365 mg, 1.7384 mmol). The reaction mixture was heated to 80° C. for 22 hours, allowed to cool to room temperature, diluted with H₂O and extracted with ethyl acetate (×3). The combined organic extracts were absorbed onto silica gel and purified by flash chromatography on silica gel using a gradient of 0-70% ethyl acetate in cyclohexane as an eluent to give 5-chloro-2-[[4-(trifluoromethyl)imidazol-1-yl]methyl]pyrimidine I7 (275 mg, 68%) as a yellow solid. ¹H NMR (400 MHz, DMSO-d₆) δ=8.96 (s, 2H), 7.94 (s, 1H), 7.88 (s, 1H), 5.58 (s, 2H)

### Step 2: Synthesis of 5-chloro-2-[[2-(3-methylimidazol-4-yl)-4-(trifluoromethyl)imidazol-1-yl]methyl]pyrimidine (1.013)

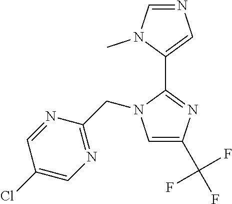 [SMILES: Cn1cncc1-c1nc(C(F)(F)F)cn1Cc1ncc(Cl)cn1]

To a solution of 5-chloro-2-[[4-(trifluoromethyl)imidazol-1-yl]methyl]pyrimidine I7 (149 mg, 0.53899 mmol) in toluene (4 mL) under an atmosphere of N₂ was added palladium(II) acetate (7 mg, 0.0296 mmol), 5-bromo-1-methyl-1H-imidazole (140 mg, 0.843473 mmol), copper(I) iodide (209 mg, 1.0974 mmol), DBU (0.17 mL, 1.1 mmol) and triphenylphosphine (15 mg, 0.0560 mmol). The reaction mixture was subjected to microwave heating at 150° C. for 4 hours, allowed to cool to room temperature, diluted with H₂O and extracted with ethyl acetate (×3). The combined organic extracts were absorbed onto silica gel and purified by flash chromatography on silica gel using a gradient of 0-50% ethyl acetate in cyclohexane as an eluent. The product-rich fractions were absorbed onto silica gel and further purified by reverse phase flash chromatography using a gradient of 40-70% MeCN in H₂O as an eluent to give 5-chloro-2-[[2-(3-methylimidazol-4-yl)-4-(trifluoromethyl)imidazol-1-yl]methyl]pyrimidine 1.013 (3.7 mg, 2%) as a yellow solid. ¹H NMR (400 MHz, CDCl₃) δ=8.61 (s, 2H), 7.39 (m, 1H), 6.99 (s, 1H), 6.92 (s, 1H), 6.08 (s, 2H), 4.11 (s, 3H)

### EXAMPLE 3: PREPARATION OF 5-CHLORO-2-[[2-(1,5-DIMETHYLPYRAZOL-4-YL)-4-(TRIFLUOROMETHYL)IMIDAZOL-1-YL]METHYL]PYRIMIDINE 1.012

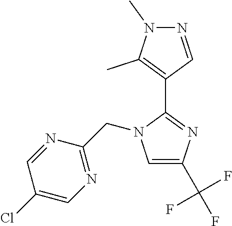 [SMILES: Cc1c(-c2nc(C(F)(F)F)cn2Cc2ncc(Cl)cn2)cnn1C]

### Step 1—Preparation of trimethyl-[2-[[4-(trifluoromethyl)imidazol-1-yl]methoxy]ethyl]silane (I8)

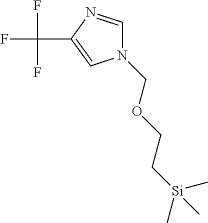 [SMILES: C[Si](C)(C)CCOCn1cnc(C(F)(F)F)c1]

To a suspension of 4-(trifluoromethyl)-1H-imidazole (3.07 g, 22.6 mmol) and K₂CO₃ (9.14 g, 66.1 mmole) in MeCN (45 mL) was added 2-(chloromethoxy)ethyl-trimethyl-silane (5.5 g, 5.8 mL, 33 mmol) and the reaction mixture stirred rapidly at room temperature under N₂ for 24 h. Additional 2-(chloromethoxy)ethyl-trimethyl-silane (1.8 g, 1.9 mL) was added dropwise to the reaction mixture and the reaction stirred for 4 h at room temperature. The reaction mixture was quenched with saturated brine (50 mL) and ethyl acetate (50 mL) was added. The phases were separated, and the aqueous phase extracted with ethyl acetate (3×50 mL). The organics were combined, washed with saturated brine and concentrated onto granulated celite. The crude product was purified by flash chromatography on silica gel using a gradient of 0-60% ethyl acetate in cyclohexane as eluent to give trimethyl-[2-[[4-(trifluoromethyl)imidazol-1-yl]methoxy]ethyl]silane (I8) (2.74 g, 46%) as a colourless oil. ¹H NMR (400 MHz, CDCl₃) δ=7.65 (s, 1H), 7.42-7.36 (m, 1H), 5.31 (s, 2H), 3.63-3.34 (m, 2H), 1.02-0.83 (m, 2H), 0.01 (s, 9H)

### Step 2—Preparation of 2-[[2-iodo-4-(trifluoromethyl)imidazol-1-yl]methoxy]ethyl-trimethyl-silane (I9)

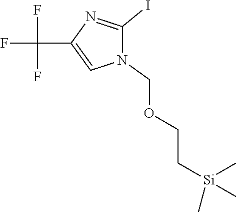 [SMILES: C[Si](C)(C)CCOCn1cc(C(F)(F)F)nc1I]

A solution of trimethyl-[2-[[4-(trifluoromethyl)imidazol-1-yl]methoxy]ethyl]silane (I8) (1.00 g, 3.76 mmol) in THF (20 mL) was cooled to an internal temperature of −70° C. under N₂. n-BuLi (2.5 M in hexanes) (2.00 mL, 5.0 mmol) was added dropwise maintaining a constant temperature below-60° C. The reaction mixture was then stirred at −70° C. for 0.5 h. To the reaction mixture was added I₂ (1.25 g, 4.92 mmol) in a single portion. The reaction mixture was stirred at −70° C. for 1 h then warmed to room temperature and stirred overnight. The reaction mixture was quenched with 2M sodium thiosulphate (25 mL) and ethyl acetate (25 mL) was added. The phases were separated and the aqueous was extracted with ethyl acetate (3×25 mL). The organics were combined and washed with saturated brine (25 mL). The organics were concentrated onto granulated celite and the crude product was purified by flash chromatography on silica gel using a gradient of 0-50% ethyl acetate in cyclohexane as eluent to give 2-[[2-iodo-4-(trifluoromethyl)imidazol-1-yl]methoxy]ethyl-trimethyl-silane (I9) (0.91 g, 62%) as a yellow solid. ¹H NMR (400 MHz, CDCl₃) δ=7.48-7.42 (m, 1H), 5.54-5.13 (m, 2H), 3.66-3.47 (m, 2H), 1.02-0.76 (m, 2H), 0.00 (d, 9H)

### Step 3—Preparation of 2-iodo-4-(trifluoromethyl)-1H-imidazole hydrochloride I10

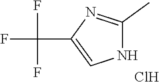

To a solution of 2-[[2-iodo-4-(trifluoromethyl)imidazol-1-yl]methoxy]ethyl-trimethyl-silane I9 (0.50 g, 1.27 mmol) in EtOH (6 mL) at room temperature was added HCl (6 M) (2.00 mL, 12.00 mmol) and the reaction heated to 60° C. under N₂ for 3.5 h. The reaction mixture was concentrated under vacuum to give 2-iodo-4-(trifluoromethyl)-1H-imidazole;hydrochloride I10 (0.348 g, 91% Yield) as a white solid. ¹H NMR (400 MHz, CD₃OD) δ=7.83 (q, 1H)

### Step 4—Preparation of 5-chloro-2-[[2-iodo-4-(trifluoromethyl)imidazol-1-yl]methyl]pyrimidine I11

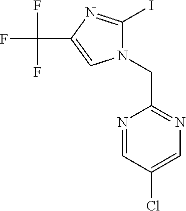 [SMILES: FC(F)(F)c1cn(Cc2ncc(Cl)cn2)c(I)n1]

To a stirred suspension of 2-iodo-4-(trifluoromethyl)-1H-imidazole hydrochloride I10 (0.35 g, 1.17 mmol) and KI (0.037 g, 0.22 mmol) in MeCN (5.25 mL) was added K₂CO₃ (0.54 g, 3.88 mmol) and H₂O (0.18 mL). 5-chloro-2-(chloromethyl)pyrimidine hydrochloride (0.28 g, 1.33 mmol) was added and the reaction was heated to 80° C. for 1.5 h. The reaction was cooled to room temperature and concentrated onto granulated celite. The crude product was purified by flash chromatography on silica gel using a gradient of 0-100% ethyl acetate in cyclohexane as eluent to give 5-chloro-2-[[2-iodo-4-(trifluoromethyl)imidazol-1-yl]methyl]pyrimidine I11 (0.38 g, 84%) as a yellow solid. ¹H NMR (400 MHz, CDCl₃) δ=8.69 (s, 2H), 7.51 (d, 1H), 5.34 (s, 2H)

### Step 5—Preparation of 5-chloro-2-[[2-(1,5-dimethylpyrazol-4-yl)-4-(trifluoromethyl)imidazol-1-yl]methyl]pyrimidine (1.012)

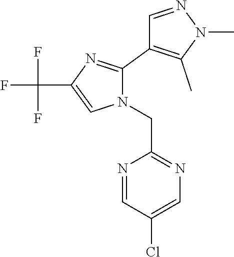 [SMILES: Cc1c(-c2nc(C(F)(F)F)cn2Cc2ncc(Cl)cn2)cnn1C]

To a 2-5 mL microwave vial was added 5-chloro-2-[[2-iodo-4-(trifluoromethyl)imidazol-1-yl]methyl]pyrimidine I11 (0.13 g, 0.33 mmol), 1,5-dimethyl-4-(4,4,5,5-tetramethyl-1,3,2-dioxaborolan-2-yl)-1H-pyrazole (0.10 g, 0.46 mmol) and (1,1′-Bis(diphenylphosphino)ferrocene)palladium(II) dichloride (13 mg, 0.016 mmol). The vessel placed under an atmosphere of N₂ and sealed. THF (2.00 mL) and potassium phosphate tribasic (1.0 M in Water) (1.00 mL, 1.00 mmol) were added and heated to 80° C. for 1 h under microwave irradiation. The reaction was cooled to room temperature and water and ethyl acetate were added. The phases were separated, and the organics were combined and concentrated onto celite. The crude product was purified by flash chromatography on silica gel using a gradient of 0-100% ethyl acetate/cyclohexane as eluent to give 5-chloro-2-[[2-(1,5-dimethylpyrazol-4-yl)-4-(trifluoromethyl)imidazol-1-yl]methyl]pyrimidine 1.012 (97 mg, 81%) as an off-white solid. ¹H NMR (400 MHz, CDCl₃) δ=8.70 (s, 2H), 7.61 (s, 1H), 7.43 (q, 1H), 5.33 (s, 2H), 3.83 (s, 3H), 2.43 (s, 3H)

### EXAMPLE 4: PREPARATION OF 5-CHLORO-2-[[2-(5-CHLORO-3-THIENYL)-4-(DIFLUOROMETHYL)IMIDAZOL-1-YL]METHYL]PYRIMIDINE 1.064

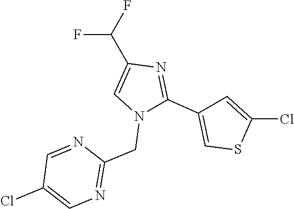 [SMILES: FC(F)c1cn(Cc2ncc(Cl)cn2)c(-c2csc(Cl)c2)n1]

### Step 1—Preparation of 1-(2-trimethylsilyloxyethyl)imidazole-4-carbaldehyde (I12)

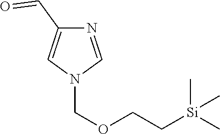

To a solution of 1H-imidazole-4-carbaldehyde (10.0 g, 104 mmol) in tetrahydrofuran (104 mL) was added sodium hydride (5.0 g, 125 mmol, 60% in mineral oil) at 0° C. The mixture was stirred at 0° C. for 15 min and 2-(trimethylsilyl)ethoxymethyl chloride (22.5 mL, 114 mmol) was added, and then the mixture was stirred at 25° C. for 10 h. The reaction mass cooled to 0° C. and quenched with saturated ammonium chloride solution. This was extracted with ethyl acetate (3×200 mL). The combined organic phase was washed with brine (100 mL), dried with anhydrous Na₂SO₄, filtered, and concentrated in to afford 1-(2-trimethylsilyloxyethyl)imidazole-4-carbaldehyde I12 (23 g, 100%) as a colourless oil. The compound was obtained as a mixture of isomers (2:1) and the mixture was taken to the next step without further purification. ¹H NMR for major isomer (400 MHz, CDCl₃) δ=9.91 (s, 1H), 7.74 (s, 1H), 7.69 (s, 1H), 5.34 (s, 2H), 3.46-3.61 (m, 4H),-0.02 (m, 9H).

### Step 2-Preparation of 2-bromo-1-(2-trimethylsilylethoxymethyl)imidazole-4-carbaldehyde (I13)

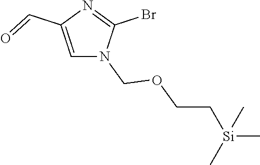 [SMILES: C[Si](C)(C)CCOCn1cc(C=O)nc1Br]

To a stirred solution of 1-(2-trimethylsilylethoxymethyl) imidazole-4-carbaldehyde I12 (2.5 g, 11 mmol) in carbontetrachloride (50 mL) was added azobisisobutyronitrile (0.093 g, 0.55 mmol). To this was added N-bromosuccinimide (2.2 g, 12 mmol) at room temperature and was heated at 90° C. for 30 h. The reaction mass was quenched with sodium thiosulphate solution, and this was extracted with ethyl acetate (3×200 ml). The combined organic layer was washed with brine solution, dried over anhydrous Na₂SO₄, filtered, and concentrated to get crude material. The crude was purified by silica column chromatography using 30% ethyl acetate in cyclohexane to afford 2-bromo-1-(2-trimethylsilylethoxymethyl) imidazole-4-carbaldehyde I13 (3.0 g, 89%) as oil. The compound was obtained as a mixture of isomers (2:1) and the mixture was taken to next step without further purification. ¹H NMR for major isomer (400 MHz, DMSO-d₆) δ=9.66 (s, 1H), 8.38 (s, 1H), 5.38 (s, 2H), 3.57-3.52 (m, 4H),-0.04 (s, 9H).

### Step 3—Preparation of 2-bromo-1H-imidazole-4-carbaldehyde (I14)

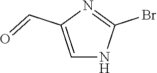

To a solution of 2-bromo-1-(2-trimethylsilylethoxymethyl)imidazole-4-carbaldehyde I13 (9.8 g, 32 mmol) in acetonitrile (98 mL) was added 2,2,2-trifluoroacetic acid (37 mL, 480 mmol). The reaction mixture was heated to 50° C. for 20 h. The reaction mixture was cooled to room temperature, concentrated in vacuo to give 2-bromo-1H-imidazole-4-carbaldehyde I14 (9.3 g, 100%) as 2,2,2-trifluoroacetate salt. The crude was used directly for the next step without further purification.

### Step 4-Preparation of 2-bromo-1-[(5-chloropyrimidin-2-yl)methyl]imidazole-4-carbaldehyde (I15)

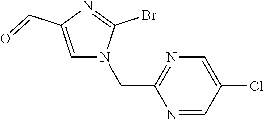 [SMILES: O=Cc1cn(Cc2ncc(Cl)cn2)c(Br)n1]

To a solution of 2-bromo-1H-imidazole-4-carbaldehyde I14 (0.4 g, 2.28 mmol) in acetonitrile (9.2 mL) was added potassium carbonate (0.63 g, 4.57 mmol) followed by 5-chloro-2-(chloromethyl)pyrimidine (0.45 g, 2.51 mmol) and potassium iodide (0.038 g, 0.23 mmol). The reaction mass was heated at 60° C. for 16 h. Reaction was cooled to room temperature, diluted with 100 ml ice cold water, extracted with ethyl acetate (4×200 mL). Organic layer was washed with brine solution (100 mL), dried over anhydrous Na₂SO₄, filtered, and concentrated to get the crude material. The crude product was purified by combi flash using ethyl acetate and cyclohexane. Desired product was eluted with 30% ethyl acetate in cyclohexane. Upon concentration, 2-bromo-1-[(5-chloropyrimidin-2-yl)methyl]imidazole-4-carbaldehyde I15 (0.455 g, 1.49 mmol, 65%) was obtained as dark yellow oil. ¹H NMR (400 MHz, CDCl₃) δ=9.81 (s, 1H), 8.69 (s, 2H), 7.81 (s, 1H), 5.41 (s, 2H).

### Step 5—Preparation of 2-[[2-bromo-4-(difluoromethyl)imidazol-1-yl]methyl]-5-chloro-pyrimidine (I16)

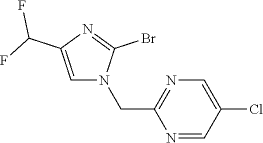 [SMILES: FC(F)c1cn(Cc2ncc(Cl)cn2)c(Br)n1]

To a solution of 2-bromo-1-[(5-chloropyrimidin-2-yl)methyl]imidazole-4-carbaldehyde I15 (0.773 g, 2.56 mmol) in dichloromethane (50 mL, 778 mmol) was added diethylaminosulfur trifluoride (3.32 g, 20.6 mmol) at 0° C. and the reaction mixture was stirred rapidly at 0° C. under nitrogen and then warmed to room temperature and stirred for 3 h. The reaction mixture was cooled to 0° C. and quenched with the slow addition of sat. NaHCO₃ (100 mL). The reaction mass was extracted with ethyl acetate (3×100 ml). The combined organic layer was washed with brine solution, dried over anhydrous Na₂SO₄, filtered, and concentrated to get the crude material. The crude product was purified by column chromatography using 20% ethyl acetate in cyclohexane to afford 2-[[2-bromo-4-(difluoromethyl)imidazol-1-yl]methyl]-5-chloro-pyrimidine I16 (0.44 g, 53%). 1H NMR (400 MHz, CDCl₃) δ=8.69 (s, 2H), 7.37 (t, 1H), 6.64 (t, 1H), 5.36 (s, 2H).

### Step 6—Preparation of 5-chloro-2-[[2-(5-chloro-3-thienyl)-4-(difluoromethyl)imidazol-1-yl]methyl]pyrimidine 1.064

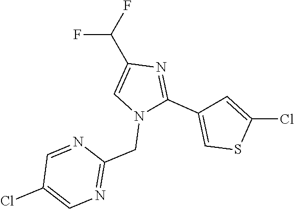 [SMILES: FC(F)c1cn(Cc2ncc(Cl)cn2)c(-c2csc(Cl)c2)n1]

To a 30 ml microwave vial, 2-[[2-bromo-4-(difluoromethyl)imidazol-1-yl]methyl]-5-chloro-pyrimidine I16 (0.25 g, 0.77mmol) in cyclopentyl methyl ether (3.75 mL) was added potassium phosphate tribasic (0.418 g, 1.93 mmol), followed by (5-chloro-3-thienyl)boronic acid (0.25 g, 1.54 mmol). The reaction mixture was purged with nitrogen for 5 min, then [1,1′-Bis-(diphenylphosphino)ferrocene]-dichloropalladium(II). Dichloromethane complex (0.031 g, 0.038 mmol) was added to it. The reaction mixture was heated at 120° C. for 2 h in microwave. Reaction mixture was cooled to room temperature and was slowly poured into ice cold solution of sodium bicarbonate. The reaction mass was extracted with ethyl acetate (3×100 ml). The combined organic layer was washed with brine solution, dried over anhydrous Na₂SO₄, filtered, and concentrated to get the crude material. The crude product was purified by column chromatography 0-100% using ethyl acetate and cyclohexane to afford 5-chloro-2-[[2-(5-chloro-3-thienyl)-4-(difluoromethyl)imidazol-1-yl]methyl]pyrimidine 1.064 (130 mg, 46%). ¹H NMR (400 MHz, CDCl₃) δ=8.74 (s, 2H), 7.58 (d, 1H), 7.38 (t, 1H), 7.36 (d, 1H),6.69 (t, 1H), 5.41 (s, 2H).

### EXAMPLE 5: PREPARATION OF 5-CHLORO-2-[[2-(5-CHLORO-2-THIENYL)-4-(DIFLUOROMETHYL)IMIDAZOL-1-YL]METHYL]PYRIMIDINE 1.058

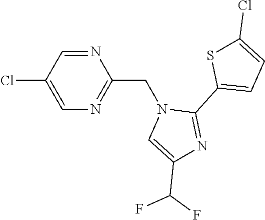 [SMILES: FC(F)c1cn(Cc2ncc(Cl)cn2)c(-c2ccc(Cl)s2)n1]

### Step 1—Preparation of ethyl 1-(2-trimethylsilylethoxymethyl)imidazole-4-carboxylate (I17)

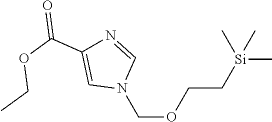 [SMILES: CCOC(=O)c1cn(COCC[Si](C)(C)C)cn1]

To a 3-necked 500 mL flask fitted with a thermometer was added sodium hydride (2.20 g, 55.0 mmol, 60%) and tetrahydrofuran (70 mL). The suspension was cooled to 0° C. under nitrogen. To this, ethyl 1H-imidazole-4-carboxylate (6.99 g, 49.9 mmol) was then added portion-wise at a rate such that the internal temperature did not exceed 8° C. When the off-gassing was completed, 2-(chloromethoxy)ethyl-trimethyl-silane (10.1 mL, 57.8 mmol) was added as a solution in tetrahydrofuran (15 mL) over 30 minutes. The reaction mixture was then allowed to warm to room temperature precipitating a white solid. The reaction mixture was stirred at room temperature under nitrogen for 3 h. The reaction mixture was quenched with sat. brine and water. Ethyl acetate (150 mL) was then added, the phases were separated, and the aqueous phase was extracted with ethyl acetate (3×100 mL). The organics were combined, washed with sat. brine and concentrated onto granulated celite. The crude product was purified by column chromatography using 0-100% ethyl acetate in cyclohexane to afford ethyl 1-(2-trimethylsilylethoxymethyl)imidazole-4-carboxylate I17 (10.4 g, 77%) as a yellow oil. ¹H NMR (400 MHz, CDCl₃) δ=7.73 (m, 1 H), 7.62 (s, 1 H), 5.31 (s, 2 H), 4.39 (m, 2 H), 3.50 (m, 2 H), 1.40 (dt, 3 H), 0.92 (m, 2 H), 0.00 (br s, 9 H).

### Step 2—Preparation of ethyl 2-bromo-1-(2-trimethylsilylethoxymethyl)imidazole-4-carboxylate (I18)

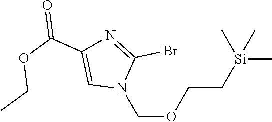 [SMILES: CCOC(=O)c1cn(COCC[Si](C)(C)C)c(Br)n1]

To a 500 mL 3-necked flask fitted with a condenser and thermometer was added ethyl 1-(2-trimethylsilylethoxymethyl)imidazole-4-carboxylate I17 (9.90 g, 36.6 mmol) and acetonitrile (100 mL) giving a pale yellow solution. The solution was degassed with a stream of nitrogen bubbled through the solution for 30 minutes. N-bromosuccinimide (8.64 g, 47.6 mmol) was added followed by azobisisobutyronitrile (0.307 g, 1.83 mmol) and the reaction mixture was heated to 65° C. under nitrogen for 6 h. The reaction was quenched with 100 mL 2 M sodium thiosulphate, and 100 mL ethyl acetate and 100 mL brine were added. The phases were separated, the aqueous layer was extracted with ethyl acetate (3×200 mL) and the organic layers were combined, dried over anhydrous Na₂SO₄, filtered, and concentrated under vacuum. The crude product was purified by column chromatography using 0-100% ethyl acetate in cyclohexane to afford ethyl 2-bromo-1-(2-trimethylsilylethoxymethyl)imidazole-4-carboxylate I18 (5.78 g, 45%) as a colourless oil. ¹H NMR (400 MHz, CDCl₃) δ=7.77 (s, 1H), 5.31 (s, 2H), 4.38 (q, 2H), 3.56 (m, 2H), 1.39 (t, 3H), 0.93 (m, 2H), 0.00 (s, 9H).

### Step 3—Preparation of ethyl 2-bromo-1H-imidazole-4-carboxylate (I19)

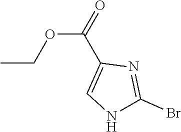

To a 500 mL flask was added ethyl 2-bromo-1-(2-trimethylsilylethoxymethyl)imidazole-4-carboxylate I18 (5.78 g, 16.5 mmol) as a solution in ethanol (145 mL) and HCl (12 M) in deionized water (14 mL, 168 mmol) was added. The reaction was heated to 60° C. under nitrogen for 1.5 h. The reaction was concentrated under vacuum to give ethyl 2-bromo-1H-imidazole-4-carboxylate I19 (4.26 g, 96%) as white solid. ¹H NMR (400 MHz, CD₃OD) δ=8.04 (s, 1H), 4.36 (q, 2H), 1.35 (t, 3H).

### Step 4—Preparation of ethyl 2-bromo-1-[(5-chloropyrimidin-2-yl)methyl]imidazole-4-carboxylate (I20)

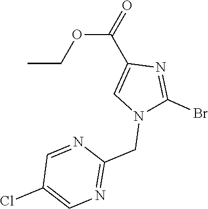 [SMILES: CCOC(=O)c1cn(Cc2ncc(Cl)cn2)c(Br)n1]

To a 100 mL flask was added ethyl 2-bromo-1H-imidazole-4-carboxylate;hydrochloride I19 (4.26 g, 15.8 mmol), potassium iodide (0.548 g, 3.30 mmol) and acetonitrile (100 mL). Potassium carbonate (6.57 g, 47.5 mmol) and water (5 mL) were added followed by 5-chloro-2-(chloromethyl)pyrimidine (2.84 g, 17.4 mmol) in a single portion. The reaction mixture was then heated to 75° C. under air for 1 h. The reaction was cooled to room temperature. The reaction was quenched with 200 mL 50% brine and 200 mL ethyl acetate was added. The phases were separated, the aqueous layer was extracted with ethyl acetate (3×200 mL) and the organic layers were combined, dried over anhydrous Na₂SO₄, filtered, and concentrated under vacuum. The crude product was purified by column chromatography using 0-100% ethyl acetate in cyclohexane to afford ethyl 2-bromo-1-[(5-chloropyrimidin-2-yl)methyl]imidazole-4-carboxylate I20 (4.45 g, 77%). ¹H NMR (400 MHz, CDCl₃) δ=8.68 (s, 2H), 7.79 (s, 1H), 5.38 (s, 2H), 4.38 (q, 2H), 1.38 (t, 3H).

### Step 5—Preparation of ethyl 1-[(5-chloropyrimidin-2-yl)methyl]-2-(5-chloro-2-thienyl)imidazole-4-carboxylate (I21)

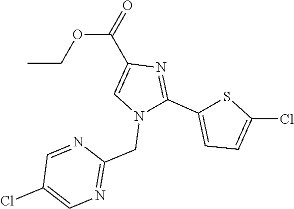 [SMILES: CCOC(=O)c1cn(Cc2ncc(Cl)cn2)c(-c2ccc(Cl)s2)n1]

To a microwave vial was added ethyl 2-bromo-1-[(5-chloropyrimidin-2-yl)methyl]imidazole-4-carboxylate I20 (0.1 g, 0.27 mmol), 5-chlorothiophene-2-boronic acid (0.053 g, 0.32 mmol), 2-methyltetrahydrofuran (2.00 mL) and potassium phosphate tribasic (1.0 M in water) (0.69 mL, 0.69 mmol). The flask was evacuated and back filled with nitrogen (three times). [1,1′-Bis(diphenylphosphino)ferrocene]dichloropalladium(II).dichloromethane complex (0.01 g, 0.012 mmol) was added to the vial and it was evacuated and back filled with nitrogen three times. The reaction was heated to 75° C. under nitrogen for 2 h. The reaction was cooled to room temperature and the reaction was quenched with 50 mL water and the phases were separated. The aqueous layer was extracted with ethyl acetate (3×50 mL) and the organic layers were combined, dried over anhydrous Na₂SO₄, filtered, and concentrated under vacuum. The crude product was purified by column chromatography using 0-100% ethyl acetate in cyclohexane to afford ethyl 1-[(5-chloropyrimidin-2-yl)methyl]-2-(5-chloro-2-thienyl)imidazole-4-carboxylate I21 (0.056 g, 56%). ¹H NMR (400 MHz, CDCl₃) δ=8.70 (s, 2H), 7.81 (s, 1H), 7.21 (d, 1H), 6.88 (d, 1H), 5.47 (s, 2H), 4.39 (q, 2H), 1.39 (t, 3H).

### Step 6—Preparation of [1-[(5-chloropyrimidin-2-yl)methyl]-2-(5-chloro-2-thienyl)imidazol-4-yl]methanol (I22)

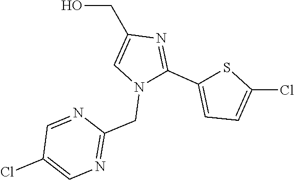 [SMILES: OCc1cn(Cc2ncc(Cl)cn2)c(-c2ccc(Cl)s2)n1]

To a 3-necked 100 mL flask equipped with a thermometer, dropping funnel and nitrogen inlet was added ethyl 1-[(5-chloropyrimidin-2-yl)methyl]-2-(5-chloro-2-thienyl)imidazole-4-carboxylate I21 (0.41 g, 1.07 mmol) and tetrahydrofuran (10 mL). The reaction was cooled to 0° C. under nitrogen and 1 M diisobutylaluminum hydridein hexane, (3.3 mL, 3.3 mmol) was added dropwise via dropping funnel maintaining a temperature less than 15° C. On complete addition, the reaction was then warmed to room temperature and stirred for 24 h. The reaction mixture was diluted with methyl tert-butyl ether (50 mL). The reaction was quenched by adding 0.131 ml of water dropwise at 0° C. followed by 0.131 mL of 15% NaOH. To this, 0.33 ml of water was then added dropwise. The reaction mixture was warmed to room temperature for 30 minutes and MgSO₄ was added. The reaction mixture was then filtered through a pad of celite. The filtrate was concentrated under vacuum to give [1-[(5-chloropyrimidin-2-yl)methyl]-2-(5-chloro-2-thienyl)imidazol-4-yl]methanol I22 (0.208 g, 56%). ¹H NMR (400 MHz, CDCl₃) δ=8.70 (s, 2H), 7.15 (d, 1H), 7.08 (s, 1H), 6.86 (d, 1H), 5.42 (s, 2H), 4.64 (br s, 2H), 2.19 (br s, 1H).

### Step 7—Preparation of 1-[(5-chloropyrimidin-2-yl)methyl]-2-(5-chloro-2-thienyl)imidazole-4-carbaldehyde (I23)

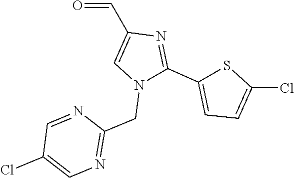 [SMILES: O=Cc1cn(Cc2ncc(Cl)cn2)c(-c2ccc(Cl)s2)n1]

To a 250 ml flask was added [1-[(5-chloropyrimidin-2-yl)methyl]-2-(5-chloro-2-thienyl)imidazol-4-yl]methanol I22 (0.208 g, 0.61 mmol) and ethyl acetate (10 mL). To the reaction mixture was added Manganese(IV) oxide (1.00 g, 11.5 mmol) and was stirred rapidly at room temperature under nitrogen overnight. The reaction was diluted with 150 mL ethyl acetate and celite was added. The suspension was then filtered through a pad and concentrated in vacuum to give 1-[(5-chloropyrimidin-2-yl)methyl]-2-(5-chloro-2-thienyl)imidazole-4-carbaldehyde I23 (0.11 g, 54%). ¹H NMR (400 MHz, CDCl₃) δ=9.92 (s, 1H), 8.71 (s, 2H), 7.83 (s, 1H), 7.22 (d, 1H), 6.90 (d, 1H), 5.51 (s, 2H).

### Step 8—Preparation of 5-chloro-2-[[2-(5-chloro-2-thienyl)-4-(difluoromethyl)imidazol-1-yl]methyl]pyrimidine 1.058

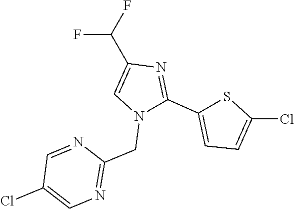 [SMILES: FC(F)c1cn(Cc2ncc(Cl)cn2)c(-c2ccc(Cl)s2)n1]

To a 250 ml flask was added 1-[(5-chloropyrimidin-2-yl)methyl]-2-(5-chloro-2-thienyl)imidazole-4-carbaldehyde I23 (0.113 g, 0.33 mmol) and dichloromethane (10 mL). The reaction was cooled to 0° C. and diethylaminosulfur trifluoride (0.25 mL, 1.9 mmol) was added and the reaction stirred rapidly at 0° C. under nitrogen for 3 h. The reaction was warmed to room temperature and was left stirring for 16 h. After this time, the reaction mixture was cooled to 0° C. and quenched with slow addition of 50 mL sat. NaHCO₃. 100 ml dichloromethane was added, and the biphasic mixture passed through a phase filter. The filtrate was concentrated to give the crude. The crude product was purified by column chromatography using 0-100% ethyl acetate in cyclohexane to afford 5-chloro-2-[[2-(5-chloro-2-thienyl)-4-(difluoromethyl)imidazol-1-yl]methyl]pyrimidine 1.058 (0.086 g, 71%). ¹H NMR (400 MHz, CDCl₃) δ=8.72 (s, 2H), 7.38 (t, 1H), 7.21 (d, 1H), 6.89 (d, 1H), 6.70 (t, 1H), 5.46 (s, 2H).

### EXAMPLE 6: PREPARATION OF 2-CHLORO-5-[1-[(5-CHLOROPYRIMIDIN-2-YL)METHYL]-4-(DIFLUOROMETHYL)IMIDAZOL-2-YL]THIAZOLE 1.071

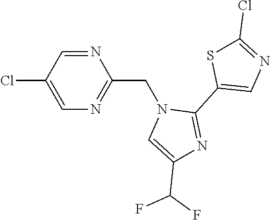 [SMILES: FC(F)c1cn(Cc2ncc(Cl)cn2)c(-c2cnc(Cl)s2)n1]

### Step 1—Preparation of ethyl 2-chloro-5-[4-(trifluoromethyl)-1H-imidazol-2-yl]thiazole (I24)

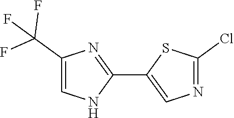 [SMILES: FC(F)(F)c1c[nH]c(-c2cnc(Cl)s2)n1]

To a 25 mL flask equipped with a reflux condenser and thermometer was added sodium acetate (1.68 g, 20.3 mmol) and water (6.00 mL). 1,1-Dibromo-3,3,3-trifluoroacetone (1.40 mL, 9.76 mmol) was added giving a turbid solution/suspension.

The reaction mixture was heated to 90° C. for 30 minutes. The reaction mixture was then cooled to room temperature to provide a solution of 1,1-dihydroxy-3,3,3-trifluoroacetone. In another 3-necked 100 mL flask was added 2-chlorothiazole-5-carbaldehyde (1.2 g, 8.131 mmol), methanol (18.00 mL) and aqueous ammonia (6.00 mL, 51 mmol) giving an orange solution. To this reaction mixture was added a solution of 1,1-dihydroxy-3,3,3-trifluoroacetone (as prepared above) dropwise over 0.5 h maintaining the internal temperature at 45° C. using a dropping funnel (Note: addition is exothermic). After complete addition, the reaction mixture was stirred at room temperature under air overnight. The reaction mixture was concentrated under vacuum to remove the MeOH and excess NH₃. The residue was diluted with Water (10 ml) and ethyl acetate (30 ml). The phases were separated, and the aqueous phase extracted with ethyl acetate (30 ml). The organics were combined, filtered through a hydrophobic filter, and concentrated. The crude product was purified by column chromatography using 0-50% ethyl acetate in cyclohexane to afford ethyl 2-chloro-5-[4-(trifluoromethyl)-1H-imidazol-2-yl]thiazole I24 (0.66 g, 32%). ¹H NMR (400 MHz, CD₃OD) δ=8.01 (s, 1H), 7.71 (s, 1H).

### Step 2—Preparation of 2-(2-chlorothiazol-5-yl)-1H-imidazole-4-carbonitrile (I25)

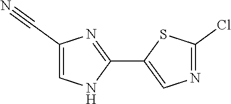 [SMILES: N#Cc1c[nH]c(-c2cnc(Cl)s2)n1]

In a vial was added 2-chloro-5-[4-(trifluoromethyl)-1H-imidazol-2-yl]thiazole I24 (0.663 g, 2.61 mmol), methanol (3.32 mL) and aqueous ammonia (6.2 mL, 39.2 mmol). The reaction mixture was stirred at 70° C. overnight. The reaction mixture was cooled down to room temperature and evaporated under reduced pressure to give a brown gum. The crude product was purified by column chromatography using 0-50% ethyl acetate in cyclohexane to afford 2-(2-chlorothiazol-5-yl)-1H-imidazole-4-carbonitrile I25 (0.33 g, 60%). ¹H NMR (400 MHz, CD₃OD) δ=8.01 (s, 1H), 8.00 (s, 1H).

### Step 3—Preparation of 2-(2-chlorothiazol-5-yl)-1H-imidazole-4-carbaldehyde (I26)

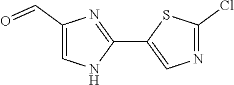 [SMILES: O=Cc1c[nH]c(-c2cnc(Cl)s2)n1]

To a stirred solution of 2-(2-chlorothiazol-5-yl)-1H-imidazole-4-carbonitrile I25 (0.320 g, 1.52 mmol) in tetrahydrofuran (4.00 mL) which was cooled to −78° C. under nitrogen was added diisobutylaluminium hydride (1.0 M in toluene) (7.60 mL, 7.60 mmol) dropwise via syringe over 10 minutes. The reaction mixture was stirred at −78° C. for 1 hour, then allowed to warm to 0° C. and stirred at 0° C. for another 1 h. The reaction was quenched at 0° C. by the addition of 0.3 ml of water, and 0.3 mL of 15% aq NaOH and finally by adding 0.76 mL of water. The reaction mixture was warmed to room temperature and stirred for 15 minutes at room temperature. Methyl tert-butyl ether (15 ml) was added to this reaction mixture followed by celite and MgSO₄. The reaction mixture was filtered, and the filter cake was washed with ethyl acetate (3×20 ml). The filtrate was concentrated under vacuum to give 66 mg of a pale orange solid. The filter cake was neutralised with 2M HCl and re-suspended in ethyl acetate (50 ml) and stirred for 2.5 h. The filtrate was concentrated under vacuum to give another 44 mg of an orange solid. The filter cake was acidified with 2 M HCl and re-suspended in ethyl acetate (50 ml) and stirred for 30 mins. The filtrate was concentrated under vacuum to give another 12 mg of an orange solid. Combined solids were dried to afford 2-(2-chlorothiazol-5-yl)-1H-imidazole-4-carbaldehyde I26 (112 mg, 34%) as an orange solid. ¹H NMR (400 MHz, CD₃OD) δ=9.65 (s, 1H), 7.98 (s, 1H), 7.88 (s, 1H).

### Step 4—Preparation of 1-[(5-chloropyrimidin-2-yl)methyl]-2-(2-chlorothiazol-5-yl)imidazole-4-carbaldehyde (I27)

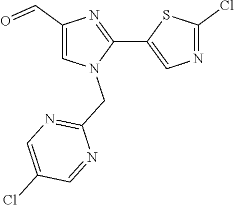 [SMILES: O=Cc1cn(Cc2ncc(Cl)cn2)c(-c2cnc(Cl)s2)n1]

To a 50 mL flask was added 2-(2-chlorothiazol-5-yl)-1H-imidazole-4-carbaldehyde I26 (0.112 g, 0.524 mmol) in acetonitrile (1.75 mL) and water (0.05 mL). Potassium carbonate (0.217 g, 1.57 mmol), 5-chloro-2-(chloromethyl) pyrimidine hydrochloride (0.132 g, 0.629 mmol) and potassium iodide (0.0174 g, 0.105 mmol), were added to the above reaction mixture giving a brown suspension. This mixture was heated to 70° C. After 1 h, another portion of 5-chloro-2-(chloromethyl)pyrimidine hydrochloride (55 mg, 0.26 mmol) and potassium carbonate (36 mg, 0.26 mmol) were added and heated for additional 45 minutes. After this time, the reaction mixture was cooled to room temperature. The reaction mixture was diluted with water (4 mL) and brine (4 mL) and extracted with ethyl acetate (2×15 mL). The combined organics were passed through a hydrophobic filter paper and concentrated in vacuo to give the crude material as a brown gum (345 mg). The crude product was purified by column chromatography using 0-100% ethyl acetate in cyclohexane to afford 1-[(5-chloropyrimidin-2-yl) methyl]-2-(2-chlorothiazol-5-yl)imidazole-4-carbaldehyde I27 (0.052 g, 29%). ¹H NMR (400 MHz, CDCl₃) δ=9.90 (s, 1H), 8.71 (s, 2H), 7.91 (s, 1H), 7.86 (s, 1H), 5.51 (s, 2H).

### Step 5—Preparation of 2-chloro-5-[1-[(5-chloropyrimidin-2-yl)methyl]-4-(difluoromethyl)imidazol-2-yl]thiazole 1.071

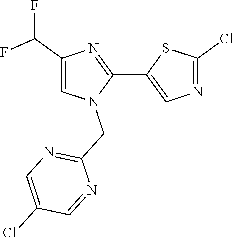 [SMILES: FC(F)c1cn(Cc2ncc(Cl)cn2)c(-c2cnc(Cl)s2)n1]

To a 50 mL 3-necked flask was added 1-[(5-chloropyrimidin-2-yl)methyl]-2-(2-chlorothiazol-5-yl)imidazole-4-carbaldehyde I27 (0.09 g, 0.2646 mmol) and dichloromethane (5.3 mL). The reaction was cooled to 0° C. in an ice/water bath and stirred under nitrogen. To the reaction mixture was added diethylaminosulfur trifluoride (0.28 mL, 2.1 mmol) and the reaction mixture was stirred rapidly at 0° C. under nitrogen for 3 h. The reaction mixture was warmed to room temperature and stirred at room temperature for additional 48 h. After this time, the reaction mixture was cooled to 0° C. and quenched with slow addition of sat. NaHCO₃ (3 ml). 50 mL dichloromethane was added, and the biphasic mixture were passed through a hydrophobic phase filter. The filtrate was concentrated to give an orange gum (105 mg). The crude product was purified by column chromatography using 40% ethyl acetate in cyclohexane to afford 2-chloro-5-[1-[(5-chloropyrimidin-2-yl)methyl]-4-(difluoromethyl)imidazol-2-yl]thiazole 1.071 (0.054 g, 56%). ¹H NMR (400 MHz, CDCl₃) δ=8.71 (s, 2H), 7.90 (s, 1H), 7.42 (t, 1H), 6.68 (t, 1H), 5.45 (s, 2H).

### EXAMPLE 7: 5-CHLORO-2-[[2-(4-CHLOROPYRAZOL-1-YL)-4-(TRIFLUOROMETHYL)IMIDAZOL-1-YL]METHYL]-PYRIMIDINE 1.065

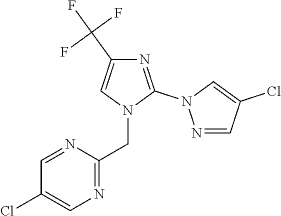 [SMILES: FC(F)(F)c1cn(Cc2ncc(Cl)cn2)c(-n2cc(Cl)cn2)n1]

To a solution of 5-chloro-2-[[2-iodo-4-(trifluoromethyl)imidazol-1-yl]methyl]pyrimidine I11 (prepared in example 3, step 4) (0.2 g, 0.51 mmol) in acetonitrile (2 mL) were added 4-chloro-1H-pyrazole (0.26 g, 2.57 mmol) followed by potassium carbonate (0.71 g, 5.14 mmol) and 8-hydroxyquinoline (0.03 g, 0.20 mmol) in 30 mL microwave vial. The reaction mixture was degassed for the 5 min by bubbling with nitrogen, then tetrakis(acetonitrile)copper(I) tetrafluoroborate (0.033 g, 0.10 mmol) was added to it and the reaction mixture was heated at 130° C. for 1.5 h. Reaction was cooled to room temperature, diluted with 70 ml water, extracted with ethyl acetate (4×80 mL), Organic layer was washed with brine solution (25 mL), dried over anhydrous Na₂SO₄, filtered and concentrated to get crude material. The crude product was purified by column chromatography using 10% ethyl acetate in cyclohexane to afford 5-chloro-2-[[2-(4-chloropyrazol-1-yl)-4-(trifluoromethyl)imidazol-1-yl]methyl]pyrimidine 1.065 (0.091 g, 48%) as white solid. ¹H NMR (400 MHz, CDCl₃) δ=8.61 (s, 2H), 8.21 (d, 1H), 7.49 (d, 1H), 7.30 (q, 1H), 5.78 (s, 2H).

TABLE 1
Compounds of the present invention

| | | ¹HNMR (400 MHz, CDCl₃ unless |
|---|---|---|
| COMPOUND | STRUCTURE | otherwise stated) |
| 1.001 | 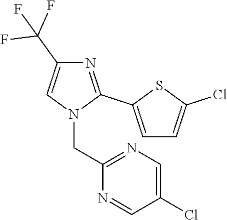 [SMILES: FC(F)(F)c1cn(Cc2ncc(Cl)cn2)c(-c2ccc(Cl)s2)n1] | δ = 8.71 (s, 2H), 7.47 (d, 1H), 7.23 (d, 1H), 6.89 (d, 1H), 5.45 (s, 2H) |
| 1.002 | 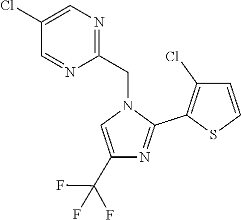 [SMILES: FC(F)(F)c1cn(Cc2ncc(Cl)cn2)c(-c2sccc2Cl)n1] | δ = 8.63 (s, 2H), 7.55 (q, 1H), 7.44 (d, 1H), 7.00 (d, 1H), 5.36 (s, 2H) |
| 1.003 | 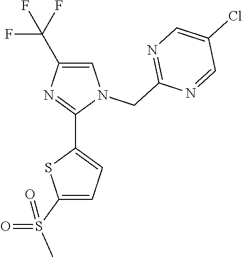 [SMILES: CS(=O)(=O)c1ccc(-c2nc(C(F)(F)F)cn2Cc2ncc(Cl)cn2)s1] | δ = 8.72 (s, 2H), 7.67 (d, 1H), 7.54 (q, 1H), 7.51 (d, 1H), 5.49 (s, 2H), 3.19 (s, 3H) |
| 1.004 | 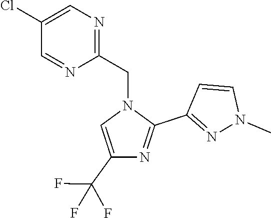 [SMILES: Cn1ccc(-c2nc(C(F)(F)F)cn2Cc2ncc(Cl)cn2)n1] | δ = 8.64 (s, 2H), 7.36 (q, 1H), 7.33 (d, 1H), 6.88 (d, 1H), 5.94 (s, 2H), 3.82 (s, 3H) |
| 1.005 | 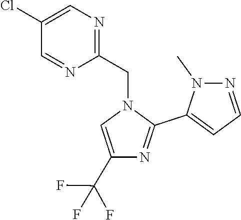 [SMILES: Cn1nccc1-c1nc(C(F)(F)F)cn1Cc1ncc(Cl)cn1] | δ = 8.69 (s, 2H), 7.52 (q, 1H), 7.51 (d, 1H), 6.51 (d, 1H), 5.39 (s, 2H), 4.05 (s, 3H) |
| 1.006 | 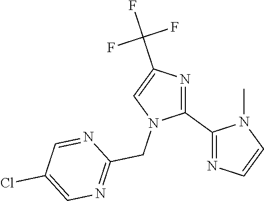 [SMILES: Cn1ccnc1-c1nc(C(F)(F)F)cn1Cc1ncc(Cl)cn1] | δ = 8.61 (s, 2H), 7.38 (q, 1H), 6.98 (d, 1H), 6.91 (d, 1H), 6.08 (s, 2H), 4.07 (s, 3H) |
| 1.007 | 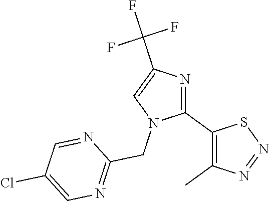 [SMILES: Cc1nnsc1-c1nc(C(F)(F)F)cn1Cc1ncc(Cl)cn1] | δ = 8.68 (s, 2H), 7.55 (q, 1H), 5.31 (s, 2H), 2.83 (s, 3H) |
| 1.008 | 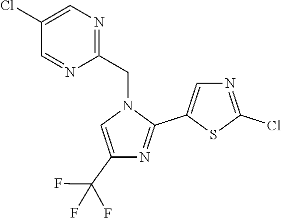 [SMILES: FC(F)(F)c1cn(Cc2ncc(Cl)cn2)c(-c2cnc(Cl)s2)n1] | δ = 8.72 (s, 2H), 7.93 (s, 1H), 7.52 (d, 1H), 5.46 (s, 2H) |
| 1.009 | 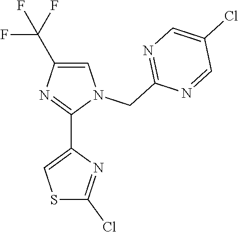 [SMILES: FC(F)(F)c1cn(Cc2ncc(Cl)cn2)c(-c2csc(Cl)n2)n1] | δ = 8.62 (s, 2H), 7.95 (s, 1H), 7.36 (d, 1H), 5.93 (s, 2H) |
| 1.010 | 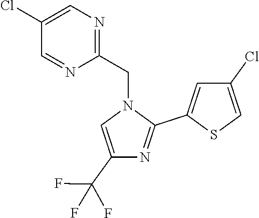 [SMILES: FC(F)(F)c1cn(Cc2ncc(Cl)cn2)c(-c2cc(Cl)cs2)n1] | δ = 8.73 (s, 2H), 7.48 (d, 1H), 7.36 (d, 1H), 7.21 (d, 1H), 5.47 (s, 2H) |
| 1.011 | 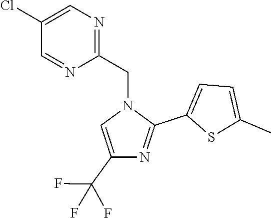 [SMILES: Cc1ccc(-c2nc(C(F)(F)F)cn2Cc2ncc(Cl)cn2)s1] | δ = 8.70 (s, 2H), 7.44 (s, 1H), 7.20 (d, 1H), 6.72 (d, 1H), 5.47 (s, 2H), 2.49 (d, 3H) |
| 1.012 | 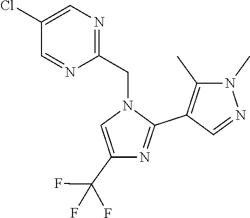 [SMILES: Cc1c(-c2nc(C(F)(F)F)cn2Cc2ncc(Cl)cn2)cnn1C] | δ = 8.70 (s, 2H), 7.61 (s, 1H), 7.43 (q, 1H), 5.33 (s, 2H), 3.83 (s, 3H), 2.43 (s, 3H) |
| 1.013 | 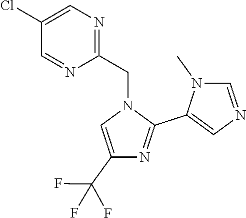 [SMILES: Cn1cncc1-c1nc(C(F)(F)F)cn1Cc1ncc(Cl)cn1] | δ = 8.61 (s, 2H), 7.39 (d, 1H), 6.99 (s, 1H), 6.92 (s, 1H), 6.08 (s, 2H), 4.11 (s, 3H) |
| 1.014 | 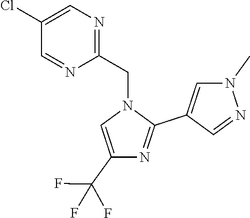 [SMILES: Cn1cc(-c2nc(C(F)(F)F)cn2Cc2ncc(Cl)cn2)cn1] | δ = 8.72 (s, 2H), 7.90 (s, 1H), 7.83 (d, 1H), 7.45 (q, 1H), 5.39 (s, 2H), 3.95 (s, 3H) |
| 1.015 | 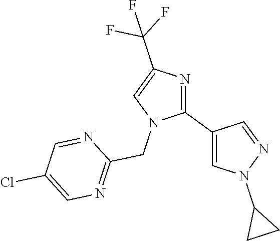 [SMILES: FC(F)(F)c1cn(Cc2ncc(Cl)cn2)c(-c2cnn(C3CC3)c2)n1] | δ = 8.71 (s, 2H), 7.99 (s, 1H), 7.81 (d, 1H), 7.44 (q, 1H), 5.39 (s, 2H), 3.64 (tt, 1H), 1.19-1.11 (m, 2H), 1.09-1.01 (m, 2H) |
| 1.016 | 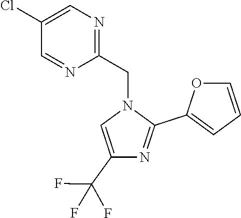 [SMILES: FC(F)(F)c1cn(Cc2ncc(Cl)cn2)c(-c2ccco2)n1] | δ = 8.66 (s, 2H), 7.41 (m, 2H), 6.99 (dd, 1H), 6.48 (dd, 1H), 5.64 (s, 2H) |
| 1.017 | 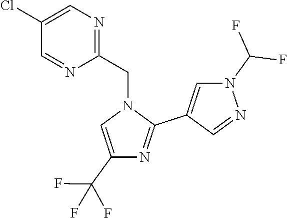 [SMILES: FC(F)n1cc(-c2nc(C(F)(F)F)cn2Cc2ncc(Cl)cn2)cn1] | δ = 8.74 (s, 2H), 8.43 (s, 1H), 8.11 (s, 1H), 7.51 (q, 1H), 7.23 (t, 1H), 5.39 (s, 2H) |
| 1.018 | 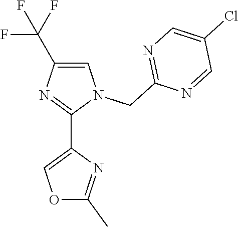 [SMILES: Cc1nc(-c2nc(C(F)(F)F)cn2Cc2ncc(Cl)cn2)co1] | δ = 8.64 (s, 2H), 8.17 (s, 1H), 7.35 (s, 1H), 5.93 (s, 2H), 2.41 (s, 3H) |
| 1.019 | 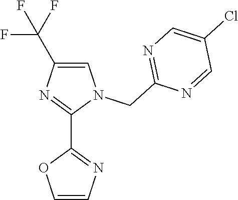 [SMILES: FC(F)(F)c1cn(Cc2ncc(Cl)cn2)c(-c2ncco2)n1] | δ = 8.63 (s, 2H), 7.73 (d, 1H), 7.49 (d, 1H), 7.17 (d, 1H), 6.06 (s, 2H) |
| 1.020 | 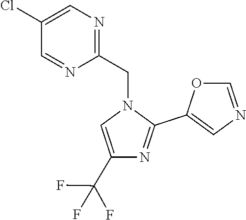 [SMILES: FC(F)(F)c1cn(Cc2ncc(Cl)cn2)c(-c2cnco2)n1] | δ = 8.67 (s, 2H), 7.91 (s, 1H), 7.66 (s, 1H), 7.50 (d, 1H), 5.58 (s, 2H) |
| 1.021 | 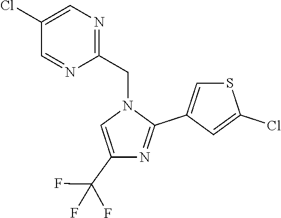 [SMILES: FC(F)(F)c1cn(Cc2ncc(Cl)cn2)c(-c2csc(Cl)c2)n1] | δ = 8.74 (s, 2H), 7.61 (d, 1H), 7.47 (s, 1H), 7.37 (d, 1H), 5.41 (s, 2H) |
| 1.022 | 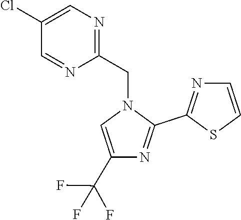 [SMILES: FC(F)(F)c1cn(Cc2ncc(Cl)cn2)c(-c2nccs2)n1] | δ = 8.62 (s, 2H), 7.72 (d, 1H), 7.44 (d, 1H), 7.36 (d, 1H), 6.11 (s, 2H) |
| 1.023 | 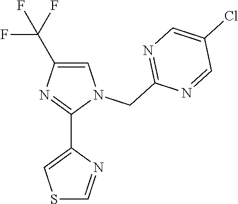 [SMILES: FC(F)(F)c1cn(Cc2ncc(Cl)cn2)c(-c2cscn2)n1] | δ = 8.71 (d, 1H), 8.61 (s, 2H), 8.15 (d, 1H), 7.40 (d, 1H), 6.04 (s, 2H) |
| 1.024 | 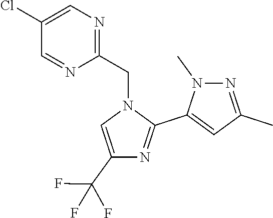 [SMILES: Cc1cc(-c2nc(C(F)(F)F)cn2Cc2ncc(Cl)cn2)n(C)n1] | δ = 8.70 (s, 2H), 7.49 (d, 1H), 6.26 (s, 1H), 5.40 (s, 2H), 3.97 (s, 3H), 2.27 (s, 3H) |
| 1.025 | 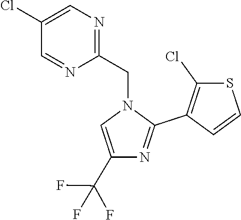 [SMILES: FC(F)(F)c1cn(Cc2ncc(Cl)cn2)c(-c2ccsc2Cl)n1] | δ = 8.65 (s, 2H), 7.50 (d, 1H), 7.16 (d, 1H), 7.06 (d, 1H), 5.30 (s, 2H) |
| 1.026 | 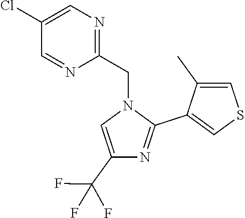 [SMILES: Cc1cscc1-c1nc(C(F)(F)F)cn1Cc1ncc(Cl)cn1] | |
| 1.027 | 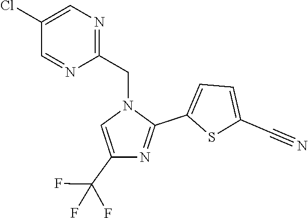 [SMILES: N#Cc1ccc(-c2nc(C(F)(F)F)cn2Cc2ncc(Cl)cn2)s1] | (400 MHz, DMSO-d6) δ = 8.96 (s, 2H), 8.17 (d, 1H), 7.91 (d, 1H), 7.39 (d, 1H), 5.83 (s, 2H) |
| 1.028 | 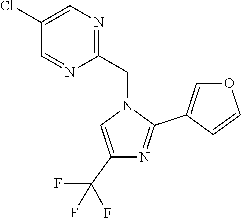 [SMILES: FC(F)(F)c1cn(Cc2ncc(Cl)cn2)c(-c2ccoc2)n1] | |
| 1.029 | 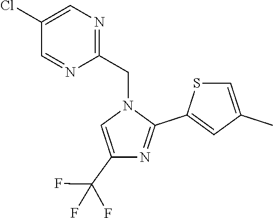 [SMILES: Cc1csc(-c2nc(C(F)(F)F)cn2Cc2ncc(Cl)cn2)c1] | δ = 8.71 (s, 2H), 7.44 (d, 1H), 7.24 (s, 1H), 6.99 (s, 1H), 5.50 (s, 2H), 2.26 (d, 3H) |
| 1.030 | 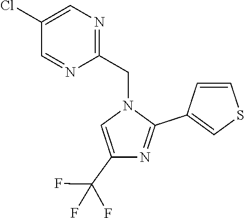 [SMILES: FC(F)(F)c1cn(Cc2ncc(Cl)cn2)c(-c2ccsc2)n1] | δ = 8.73 (s, 2H), 7.80 (dd, 1H), 7.49 (dd, 2H), 7.40 (dd, 1H), 5.43 (s, 2H) |
| 1.032 | 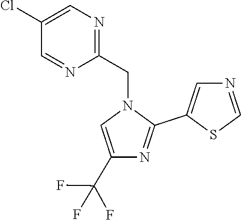 [SMILES: FC(F)(F)c1cn(Cc2ncc(Cl)cn2)c(-c2cncs2)n1] | |
| 1.033 | 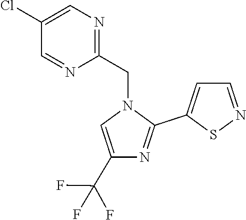 [SMILES: FC(F)(F)c1cn(Cc2ncc(Cl)cn2)c(-c2ccns2)n1] | δ = 8.72 (s, 2H), 8.49 (d, 1H), 7.65 (d, 1H), 7.57 (d, 1H), 5.50 (s, 2H) |
| 1.034 | 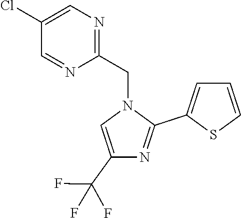 [SMILES: FC(F)(F)c1cn(Cc2ncc(Cl)cn2)c(-c2cccs2)n1] | δ = 8.71 (s, 2H), 7.48 (d, 1H), 7.43 (s, 1H), 7.42 (q, 1H), 7.08 (dd, 1H), 5.49 (s, 2H) |
| 1.035 | 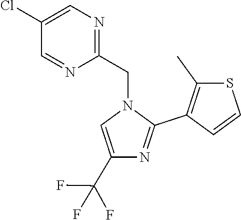 [SMILES: Cc1sccc1-c1nc(C(F)(F)F)cn1Cc1ncc(Cl)cn1] | δ = 8.67 (s, 2H), 7.45 (s, 1H), 7.10 (d, 1H), 7.02 (d, 1H), 5.26 (s, 2H), 2.47 (s, 3H) |
| 1.036 | 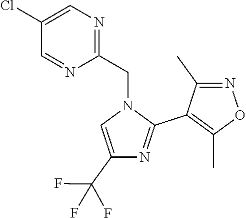 [SMILES: Cc1noc(C)c1-c1nc(C(F)(F)F)cn1Cc1ncc(Cl)cn1] | |
| 1.037 | 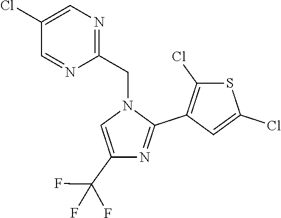 [SMILES: FC(F)(F)c1cn(Cc2ncc(Cl)cn2)c(-c2cc(Cl)sc2Cl)n1] | δ = 8.67 (s, 2H), 7.48 (d, 1H), 6.96 (s, 1H), 5.31 (s, 2H) |
| 1.038 | 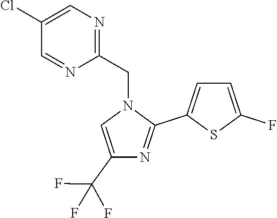 [SMILES: Fc1ccc(-c2nc(C(F)(F)F)cn2Cc2ncc(Cl)cn2)s1] | δ = 8.72 (s, 2H), 7.46 (s, 1H), 7.07 (dd, 1H), 6.47 (dd, 1H), 5.45 (s, 2H) |
| 1.039 | 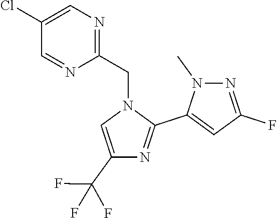 [SMILES: Cn1nc(F)cc1-c1nc(C(F)(F)F)cn1Cc1ncc(Cl)cn1] | |
| 1.040 | 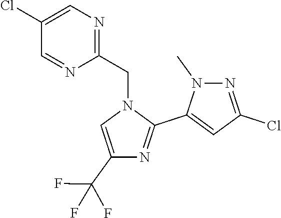 [SMILES: Cn1nc(Cl)cc1-c1nc(C(F)(F)F)cn1Cc1ncc(Cl)cn1] | δ = 8.70 (s, 2H), 7.52 (d, 1H), 6.50 (s, 1H), 5.39 (s, 2H), 4.00 (s, 3H) |
| 1.041 | 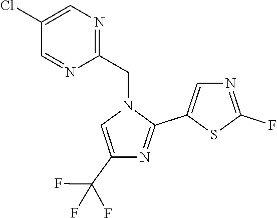 [SMILES: Fc1ncc(-c2nc(C(F)(F)F)cn2Cc2ncc(Cl)cn2)s1] | |
| 1.042 | 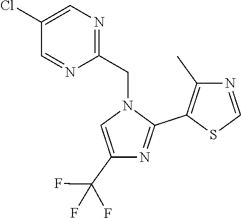 [SMILES: Cc1ncsc1-c1nc(C(F)(F)F)cn1Cc1ncc(Cl)cn1] | δ = 8.89 (s, 1H), 8.66 (s, 2H), 7.53 (d, 1H), 5.29 (s, 2H), 2.44 (s, 3H), |
| 1.043 | 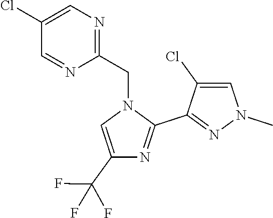 [SMILES: Cn1cc(Cl)c(-c2nc(C(F)(F)F)cn2Cc2ncc(Cl)cn2)n1] | δ = 8.63 (s, 2H), 7.44 (d, 1H), 7.41 (s, 1H), 5.70 (s, 2H), 3.81 (s, 3H) |
| 1.044 | 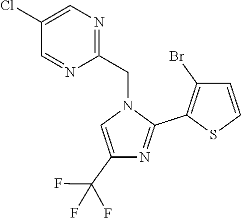 [SMILES: FC(F)(F)c1cn(Cc2ncc(Cl)cn2)c(-c2sccc2Br)n1] | δ = 8.63 (s, 2H), 7.55 (s, 1H), 7.43 (d, 1H), 7.05 (d, 1H), 5.35 (s, 2H), |
| 1.045 | 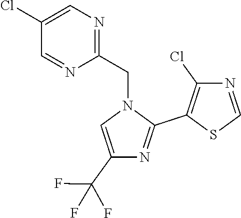 [SMILES: FC(F)(F)c1cn(Cc2ncc(Cl)cn2)c(-c2scnc2Cl)n1] | δ = 8.84 (s, 1H), 8.64 (s, 2H), 7.56 (d, 1H), 5.37 (s, 2H) |
| 1.046 | 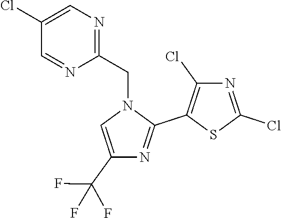 [SMILES: FC(F)(F)c1cn(Cc2ncc(Cl)cn2)c(-c2sc(Cl)nc2Cl)n1] | δ = 8.65 (s, 2H), 7.53 (s, 1H), 5.38 (s, 2H) |
| 1.047 | 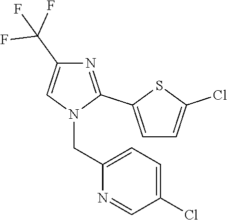 [SMILES: FC(F)(F)c1cn(Cc2ccc(Cl)cn2)c(-c2ccc(Cl)s2)n1] | δ = 8.58 (d, 1H), 7.70 (dd, 1H), 7.38 (d, 1H), 7.06 (d, 1H), 6.97 (d, 1H), 6.87 (d, 1H), 5.37 (s, 2H) |
| 1.048 | 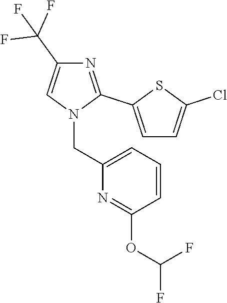 [SMILES: FC(F)Oc1cccc(Cn2cc(C(F)(F)F)nc2-c2ccc(Cl)s2)n1] | δ = 7.76 (t, 1H), 7.37 (d, 1H), 7.31 (t, 1H), 7.07 (d, 1H), 6.91 - 6.87 (m, 2H), 6.83 (d, 1H), 5.31 (s, 2H) |
| 1.049 | 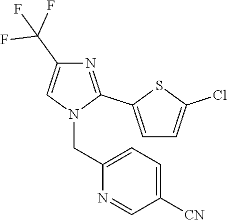 [SMILES: N#Cc1ccc(Cn2cc(C(F)(F)F)nc2-c2ccc(Cl)s2)nc1] | δ = 8.90 (d, 1H), 8.00 (dd, 1H), 7.40 (d, 1H), 7.12 (d, 1H), 7.00 (d, 1H), 6.87 (d, 1H), 5.48 (s, 2H) |
| 1.050 | 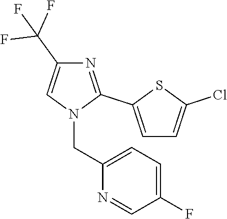 [SMILES: Fc1ccc(Cn2cc(C(F)(F)F)nc2-c2ccc(Cl)s2)nc1] | δ = 8.49 (d, 1H), 7.46 - 7.37 (m, 2H), 7.08 (d, 1H), 7.03 (dd, 1H), 6.88 (d, 1H), 5.38 (s, 2H) |
| 1.051 | 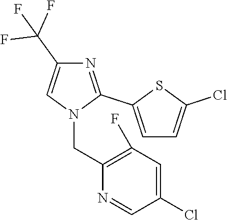 [SMILES: Fc1cc(Cl)cnc1Cn1cc(C(F)(F)F)nc1-c1ccc(Cl)s1] | δ = 8.41 (s, 1H), 7.55 (dd, 1H), 7.39 (s, 1H), 7.32 (d, 1H), 6.93 (d, 1H), 5.41 (s, 2H) |
| 1.052 | 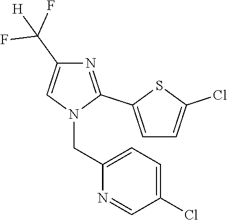 [SMILES: FC(F)c1cn(Cc2ccc(Cl)cn2)c(-c2ccc(Cl)s2)n1] | |
| 1.053 | 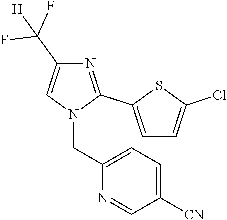 [SMILES: N#Cc1ccc(Cn2cc(C(F)F)nc2-c2ccc(Cl)s2)nc1] | |
| 1.054 | 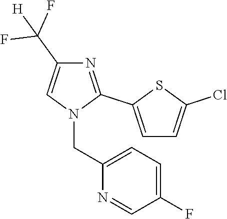 [SMILES: Fc1ccc(Cn2cc(C(F)F)nc2-c2ccc(Cl)s2)nc1] | |
| 1.055 | 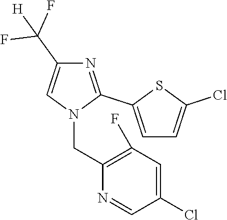 [SMILES: Fc1cc(Cl)cnc1Cn1cc(C(F)F)nc1-c1ccc(Cl)s1] | 8.41 (d, 1H), 7.55 (dd, 1H), 7.32 (m, 2H), 6.93 (d, 1H), 6.67 (t, 1H), 5.41 (d, 2H) |
| 1.056 | 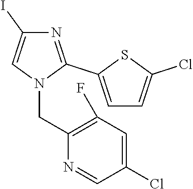 [SMILES: Fc1cc(Cl)cnc1Cn1cc(I)nc1-c1ccc(Cl)s1] | |
| 1.057 | 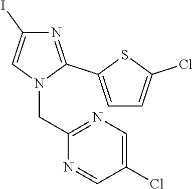 [SMILES: Clc1cnc(Cn2cc(I)nc2-c2ccc(Cl)s2)nc1] | |
| 1.058 | 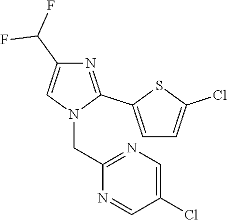 [SMILES: FC(F)c1cn(Cc2ncc(Cl)cn2)c(-c2ccc(Cl)s2)n1] | δ = 8.72 (s, 2H), 7.38 (t, 1H), 7.21 (d, 1H), 6.89 (d, 1H), 6.70 (t, 1H), 5.46 (s, 2H) |
| 1.059 | 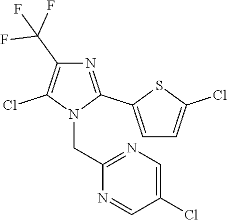 [SMILES: FC(F)(F)c1nc(-c2ccc(Cl)s2)n(Cc2ncc(Cl)cn2)c1Cl] | |
| 1.060 | 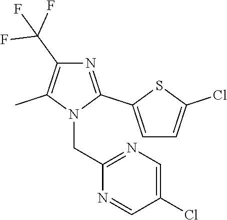 [SMILES: Cc1c(C(F)(F)F)nc(-c2ccc(Cl)s2)n1Cc1ncc(Cl)cn1] | |
| 1.061 | 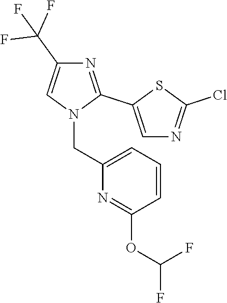 [SMILES: FC(F)Oc1cccc(Cn2cc(C(F)(F)F)nc2-c2cnc(Cl)s2)n1] | |
| 1.062 | 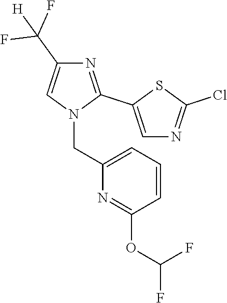 [SMILES: FC(F)Oc1cccc(Cn2cc(C(F)F)nc2-c2cnc(Cl)s2)n1] | |
| 1.063 | 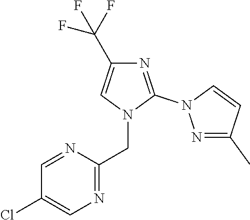 [SMILES: Cc1ccn(-c2nc(C(F)(F)F)cn2Cc2ncc(Cl)cn2)n1] | 8.61 (s, 2H), 8.07 (br s, 1H), 7.28 (s, 1H), 6.16 (br s, 1H), 5.81 (s, 2H), 2.19 (s, 3H) |
| 1.064 | 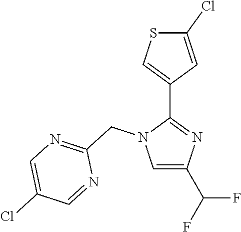 [SMILES: FC(F)c1cn(Cc2ncc(Cl)cn2)c(-c2csc(Cl)c2)n1] | 8.74 (s, 2H), 7.58 (d, 1H) 7.38 (t, 1H), 7.36 (d, 1H), 6.69, (t, 1H), 5.41 (s, 2H) |
| 1.065 | 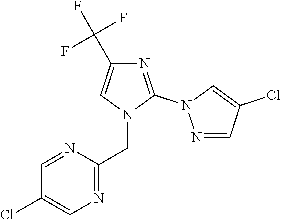 [SMILES: FC(F)(F)c1cn(Cc2ncc(Cl)cn2)c(-n2cc(Cl)cn2)n1] | 8.61 (s, 2 H), 8.21 (d, 1H), 7.49 (d, 1H), 7.30 (q, 1H), 5.78 (s, 2H) |
| 1.066 | 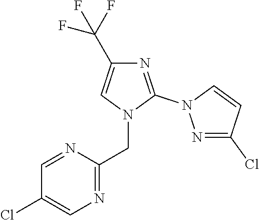 [SMILES: FC(F)(F)c1cn(Cc2ncc(Cl)cn2)c(-n2ccc(Cl)n2)n1] | 8.62 (s, 2H), 8.14 (d, 1H), 7.30 (s, 1H), 6.33 (d, 1H) 5.78 (s, 2H) |
| 1.067 | 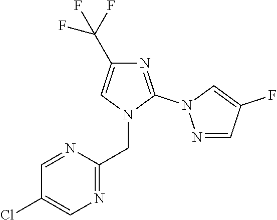 [SMILES: Fc1cnn(-c2nc(C(F)(F)F)cn2Cc2ncc(Cl)cn2)c1] | 8.61 (s, 2H), 8.08 (dd, 1H), 7.43 (d, 1H), 7.30 (q, 1H), 5.78 (s, 2H) |
| 1.068 | 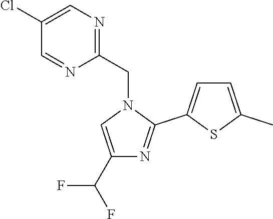 [SMILES: Cc1ccc(-c2nc(C(F)F)cn2Cc2ncc(Cl)cn2)s1] | 8.70 (s, 2H), 7.35 (d, 1H), 7.26 (s, 1H), 7.17 (d, 1H), 6.71 (t, 1H), 5.47 (s, 2H), 2.49 (d, 3H) |
| 1.069 | 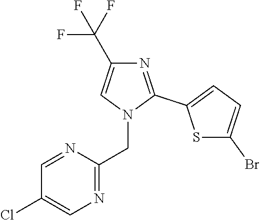 [SMILES: FC(F)(F)c1cn(Cc2ncc(Cl)cn2)c(-c2ccc(Br)s2)n1] | 8.71 (s, 2H), 7.47 (d, 1H), 7.21 (d, 1H), 7.03 (d, 1H), 5.45 (s, 2H) |
| 1.070 | 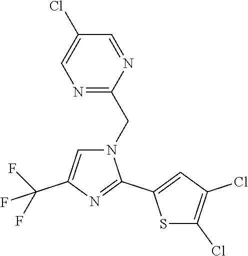 [SMILES: FC(F)(F)c1cn(Cc2ncc(Cl)cn2)c(-c2cc(Cl)c(Cl)s2)n1] | 8.73 (s, 2H), 7.48 (s, 1H), 7.32 (s, 1H), 5.45 (s, 2H) |
| 1.071 | 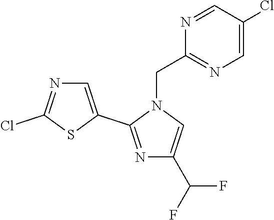 [SMILES: FC(F)c1cn(Cc2ncc(Cl)cn2)c(-c2cnc(Cl)s2)n1] | 8.71 (s, 2H), 7.90 (s, 1H), 7.42 (t, 1H), 6.68 (t, 1H), 5.45 (s, 2H) |
| 1.072 | 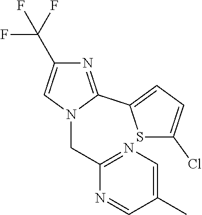 [SMILES: Cc1cnc(Cn2cc(C(F)(F)F)nc2-c2ccc(Cl)s2)nc1] | 8.58 (s, 2H), 7.48 (q, 1H), 7.29 (d, 1H), 6.88 (d, 1H), 5.42 (s, 2H), 2.35 (s, 3H) |
| 1.074 | 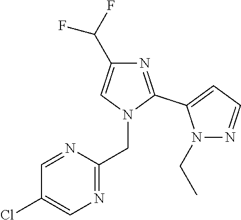 [SMILES: CCn1nccc1-c1nc(C(F)F)cn1Cc1ncc(Cl)cn1] | 8.67 (s, 2H), 7.53 (d, 1H), 7.42 (t, 1H), 6.73 (t, 1H), 6.46 (d, 1H), 5.38 (s, 2H), 4.40 (q, 2H), 1.41 (t, 3H) |
| 1.075 | 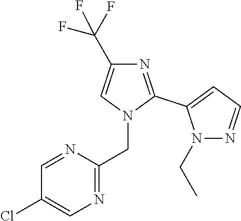 [SMILES: CCn1nccc1-c1nc(C(F)(F)F)cn1Cc1ncc(Cl)cn1] | 8.66 (s, 2H), 7.51 (m, 2H), 6.46 (d, 1H), 5.37 (s, 2H), 4.38 (q, 2H), 1.39 (t, 3H) |
| 1.076 | 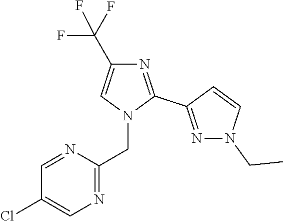 [SMILES: CCn1ccc(-c2nc(C(F)(F)F)cn2Cc2ncc(Cl)cn2)n1] | 8.61 (s, 2H), 7.34 (m, 2H), 6.78 (m, 1H), 5.92 (s, 2H), 4.04 (q, 2H), 1.35 (t, 3H) |
| 1.077 | 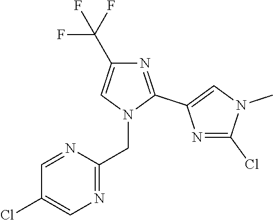 [SMILES: Cn1cc(-c2nc(C(F)(F)F)cn2Cc2ncc(Cl)cn2)nc1Cl] | 8.63 (s, 2H), 7.58 (s, 1H), 7.28 (q, 1H), 5.98 (s, 2H), 3.62 (s, 3H) |
| 1.078 | 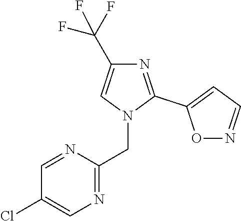 [SMILES: FC(F)(F)c1cn(Cc2ncc(Cl)cn2)c(-c2ccno2)n1] | 8.64 (s, 2H), 8.31 (d, 1H), 7.47 (s, 1H), 6.95 (d, 1H), 5.79 (s, 2H) |
| 1.079 | 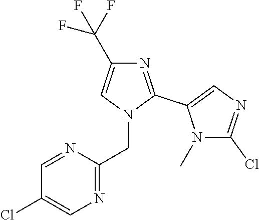 [SMILES: Cn1c(-c2nc(C(F)(F)F)cn2Cc2ncc(Cl)cn2)cnc1Cl] | 8.70 (s, 2H), 7.49 (s, 1H), 7.27(s, 1H), 5.41 (s, 2H), 3.78 (s, 3H) |
| 1.080 | 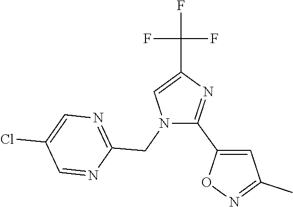 [SMILES: Cc1cc(-c2nc(C(F)(F)F)cn2Cc2ncc(Cl)cn2)on1] | 8.63 (s, 2 H), 7.44 (q, 1 H), 6.76 (s, 1 H), 5.76 (s, 2 H), 2.33 (s, 3 H) |
| 1.081 | 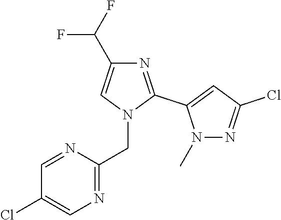 [SMILES: Cn1nc(Cl)cc1-c1nc(C(F)F)cn1Cc1ncc(Cl)cn1] | 8.69 (s, 2H), 7.42 (t, 1H), 6.70 (t, 1H), 6.48 (s, 1H), 5.37 (s, 2H), 3.98 (s, 3H) |
| 1.082 | 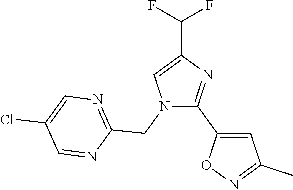 [SMILES: Cc1cc(-c2nc(C(F)F)cn2Cc2ncc(Cl)cn2)on1] | 8.62 (s, 2 H), 7.35 (t, 1 H), 6.72 (t, 1 H), 6.70 (s, 1 H), 5.74 (s, 2 H), 2.32 (s, 3 H) |

TABLE 2
Intermediate compounds of the present invention.

| | | ¹HNMR (400 MHz, CDCl₃ unless |
|---|---|---|
| COMPOUND | STRUCTURE | otherwise stated) |
| I-7 | 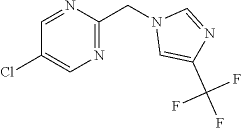 [SMILES: FC(F)(F)c1cn(Cc2ncc(Cl)cn2)cn1] | ¹H NMR (400 MHz, DMSO-d6) δ = 8.96 (s, 2H), 7.94 (s, 1H), 7.88 (s, 1H), 5.58 (s, 2H) |
| I-11 | 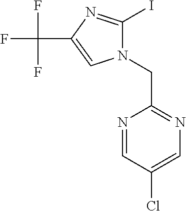 [SMILES: FC(F)(F)c1cn(Cc2ncc(Cl)cn2)c(I)n1] | δ = 8.69 (s, 2H), 7.51 (d, 1H), 5.34 (s, 2H) |
| I-16 | 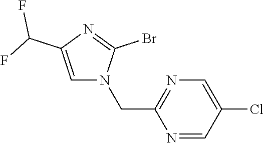 [SMILES: FC(F)c1cn(Cc2ncc(Cl)cn2)c(Br)n1] | δ = 8.69 (s, 2H), 7.37 (t, 1H), 6.64 (t, 1H), 5.36 (s, 2H) |
| I-20 | 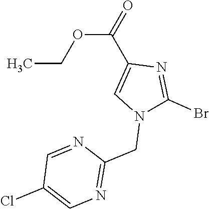 [SMILES: CCOC(=O)c1cn(Cc2ncc(Cl)cn2)c(Br)n1] | δ = 8.68 (s, 2H), 7.79 (s, 1H), 5.38 (s, 2H), 4.38 (q, 2H), 1.38 (t, 3H). |

### Biological Examples

Seeds of a variety of test species are sown in standard soil in pots Amaranthus retoflexus (AMARE), Echinochloa crus-galli (ECHCG), Setaria faberi (SETFA)). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| | Rate | | | | | |
|---|---|---|---|---|---|---|
| Compound | (g/ha) | AMARE | AMAPA | SETFA | ECHCG | IPOHE |
| 1.001 | 250 | 5 | 5 | 5 | 5 | 4 |
| 1.002 | 250 | 1 | 1 | NT | 1 | 1 |
| 1.003 | 250 | 4 | 4 | NT | 5 | 4 |
| 1.004 | 250 | 1 | 1 | NT | 1 | 1 |
| 1.005 | 250 | 5 | 5 | 5 | 5 | 4 |
| 1.006 | 250 | 1 | 1 | 1 | 1 | 1 |
| 1.007 | 250 | 5 | 4 | NT | 2 | 4 |
| 1.008 | 250 | 5 | 5 | 5 | 5 | 4 |
| 1.009 | 250 | 2 | 2 | NT | 1 | 1 |
| 1.010 | 250 | 4 | 4 | NT | 4 | 4 |
| 1.011 | 250 | 4 | 4 | NT | 4 | 2 |
| 1.012 | 250 | 2 | 3 | 1 | 1 | 3 |
| 1.014 | 250 | 3 | 3 | 2 | 2 | 3 |
| 1.015 | 250 | 5 | 4 | NT | 4 | 5 |
| 1.016 | 250 | 1 | 1 | 1 | 1 | 1 |
| 1.017 | 250 | 5 | 5 | NT | 5 | 5 |
| 1.018 | 250 | 2 | 1 | NT | 1 | 2 |
| 1.019 | 250 | 1 | 1 | NT | 1 | 1 |
| 1.020 | 250 | 3 | 2 | NT | 1 | 4 |
| 1.021 | 250 | 4 | 5 | NT | 4 | 4 |
| 1.022 | 250 | 1 | 1 | NT | 1 | 1 |
| 1.023 | 250 | 1 | 1 | NT | 1 | 2 |
| 1.024 | 250 | 5 | 5 | 5 | 5 | 5 |
| 1.025 | 250 | 4 | 3 | 4 | 4 | 3 |
| 1.027 | 250 | 5 | 5 | NT | 5 | 4 |
| 1.029 | 250 | 5 | 5 | 4 | 4 | 2 |
| 1.030 | 250 | 5 | 5 | 5 | 4 | 4 |
| 1.033 | 250 | 5 | 5 | 5 | 5 | 4 |
| 1.034 | 250 | 4 | 4 | 2 | 2 | 4 |
| 1.035 | 250 | 5 | 4 | 5 | 5 | 4 |
| 1.037 | 250 | 4 | 3 | 3 | 3 | 3 |
| 1.038 | 250 | 5 | 4 | NT | 5 | 4 |
| 1.042 | 250 | 5 | 5 | NT | 4 | 4 |
| 1.043 | 250 | 1 | 1 | NT | 1 | 1 |
| 1.044 | 250 | 2 | 2 | NT | 1 | 3 |
| 1.045 | 250 | 4 | 4 | NT | 2 | 4 |
| 1.046 | 250 | 2 | 2 | NT | 1 | 1 |
| 1.047 | 250 | 5 | 5 | NT | 4 | 4 |
| 1.048 | 250 | 1 | 1 | NT | 1 | 1 |
| 1.049 | 250 | 5 | 5 | NT | 4 | 4 |
| 1.050 | 250 | 4 | 4 | NT | 4 | 4 |
| 1.051 | 250 | 5 | 5 | NT | 4 | 3 |
| 1.058 | 250 | 5 | 5 | NT | 4 | 4 |
| 1.065 | 250 | 4 | 4 | 2 | 2 | 3 |
| 1.066 | 250 | 4 | 3 | 1 | 1 | 4 |
| 1.067 | 250 | 4 | 3 | 1 | 1 | 2 |
| 1.068 | 250 | 3 | 2 | 1 | 1 | 1 |
| 1.069 | 250 | 5 | 5 | 5 | 5 | 4 |
| 1.070 | 250 | 5 | 5 | 5 | 5 | 4 |
| 1.071 | 250 | 5 | 4 | NT | 4 | 2 |
| 1.072 | 250 | 5 | 5 | NT | 5 | 4 |

NT = Not tested

TABLE B2
Pre-emergence Test

| | Rate | | | | | |
|---|---|---|---|---|---|---|
| Compound | (g/ha) | AMARE | AMAPA | SETFA | ECHCG | IPOHE |
| 1.001 | 250 | 5 | 5 | 5 | 5 | 5 |
| 1.002 | 250 | 2 | 2 | NT | 1 | 1 |
| 1.003 | 250 | 5 | 5 | NT | 5 | 5 |
| 1.004 | 250 | 1 | 1 | NT | 1 | 1 |
| 1.005 | 250 | 5 | 5 | 5 | 5 | 5 |
| 1.006 | 250 | 1 | 1 | 1 | 1 | 1 |
| 1.007 | 250 | 5 | 5 | NT | 4 | 5 |
| 1.008 | 250 | 5 | 5 | 5 | 5 | 5 |
| 1.009 | 250 | 4 | 3 | NT | 1 | 1 |
| 1.010 | 250 | 5 | 5 | NT | 5 | 4 |
| 1.011 | 250 | 5 | 5 | NT | 5 | 5 |
| 1.012 | 250 | 3 | 5 | 1 | 1 | 5 |
| 1.014 | 250 | 4 | 5 | 4 | 4 | 5 |
| 1.015 | 250 | 5 | 5 | NT | 5 | 5 |
| 1.016 | 250 | 1 | 1 | 1 | 1 | 1 |
| 1.017 | 250 | 5 | 5 | NT | 5 | 5 |
| 1.018 | 250 | 2 | NT | NT | 1 | 1 |
| 1.019 | 250 | 1 | 1 | NT | 1 | 1 |
| 1.020 | 250 | 5 | 3 | NT | 1 | 4 |
| 1.021 | 250 | 5 | 5 | NT | 5 | 4 |
| 1.022 | 250 | NT | 1 | NT | 1 | 1 |
| 1.023 | 250 | NT | 1 | NT | 1 | 1 |
| 1.024 | 250 | 5 | 5 | 5 | 5 | 5 |
| 1.025 | 250 | 5 | 5 | 5 | 5 | 4 |
| 1.027 | 250 | 5 | 5 | NT | 5 | 5 |
| 1.029 | 250 | 5 | 5 | 5 | 5 | 1 |
| 1.030 | 250 | 5 | 5 | 5 | 5 | 4 |
| 1.033 | 250 | 5 | 5 | 5 | 5 | 5 |
| 1.034 | 250 | 5 | 5 | 2 | 2 | 2 |
| 1.035 | 250 | 5 | 5 | 5 | 5 | 4 |
| 1.037 | 250 | 5 | 5 | 4 | 4 | 5 |
| 1.038 | 250 | 5 | 5 | NT | 5 | 5 |
| 1.042 | 250 | 5 | 5 | NT | 5 | 5 |
| 1.043 | 250 | 1 | 1 | NT | 1 | 1 |
| 1.044 | 250 | 5 | 5 | NT | 1 | 1 |
| 1.045 | 250 | 5 | 5 | NT | 1 | 5 |
| 1.046 | 250 | 1 | 4 | NT | 1 | 1 |
| 1.047 | 250 | 5 | 5 | NT | 4 | 2 |
| 1.048 | 250 | 1 | 1 | NT | 1 | NT |
| 1.049 | 250 | 5 | 5 | NT | 4 | 5 |
| 1.050 | 250 | 5 | 5 | NT | 5 | 5 |
| 1.051 | 250 | 5 | 5 | NT | 5 | 4 |
| 1.058 | 250 | 5 | 5 | NT | 5 | 5 |
| 1.065 | 250 | 5 | 5 | 2 | 1 | 4 |
| 1.066 | 250 | 5 | 5 | 2 | 1 | 4 |
| 1.067 | 250 | 5 | 4 | 1 | 1 | 2 |
| 1.068 | 250 | 5 | 5 | 4 | 3 | 4 |
| 1.069 | 250 | 5 | 5 | 5 | 5 | 5 |
| 1.070 | 250 | 5 | 5 | 5 | 5 | 3 |
| 1.071 | 250 | 5 | 5 | NT | 4 | 5 |
| 1.072 | 250 | 5 | 5 | NT | 5 | 5 |

NT = Not tested

1. A compound of Formula (I): 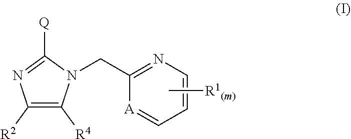
or an agronomically acceptable salt thereof,
wherein
A is CR⁵ or N;
Q is 5-membered heteroaryl which is optionally substituted by 1 or 2 R³ substituents,
R¹ is independently selected from the group consisting of halogen, —CN, C₁-C₂alkyl, C₁-C₂haloalkyl, C₃-C₆cycloalkyl, C₁-C₂alkoxy- and C₁-C₂haloalkoxy-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)ₚC₁-C₄alkyl, —C(R⁶)═NOR⁷ and C₃-C₆cycloalkyl;
R³ is independently selected from the group consisting of halogen, C₁-C₄ alkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁴ is independently selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁵ is selected from the group consisting of hydrogen, fluoro, chloro and —CN;
R⁶ is hydrogen or C₁-C₄alkyl;
R⁷ is hydrogen or C₁-C₂alkyl;
R⁸ is hydrogen or C₁-C₄alkyl;
R⁹ is hydrogen or C₁-C₄alkyl;
m=1 or 2; and
p=0, 1 or 2.

2. A compound of Formula (I) according to claim 1, which is of Formula (Ia) 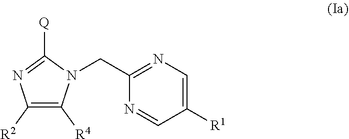
wherein Q, R¹, R² and R⁴ are as defined in claim 1 above.

3. A compound according to claim 1 wherein R⁴ is hydrogen.

4. A compound according to claim 1, wherein R¹ is chloro.

5. A compound according to claim 1, wherein R² is —CF₃ or —CF₂H.

6. A compound according to claim 1, wherein Q is selected from the group consisting of: 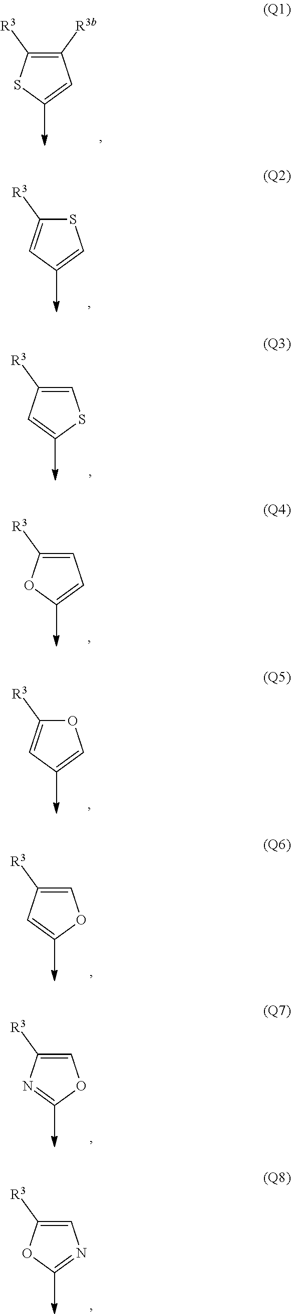 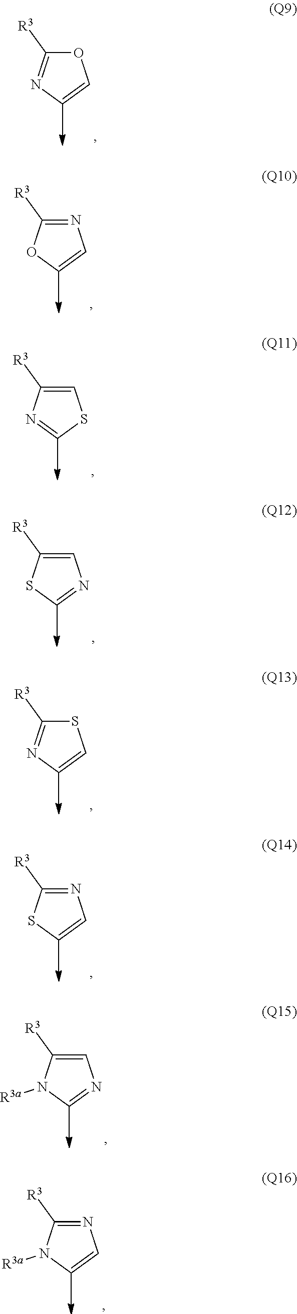 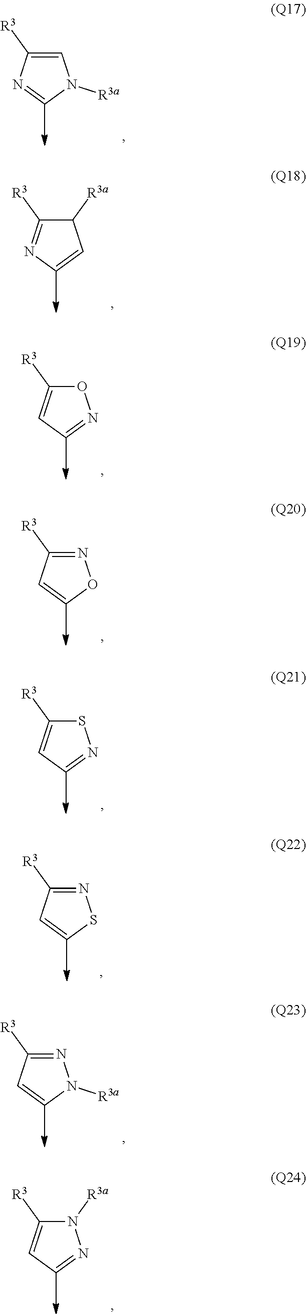 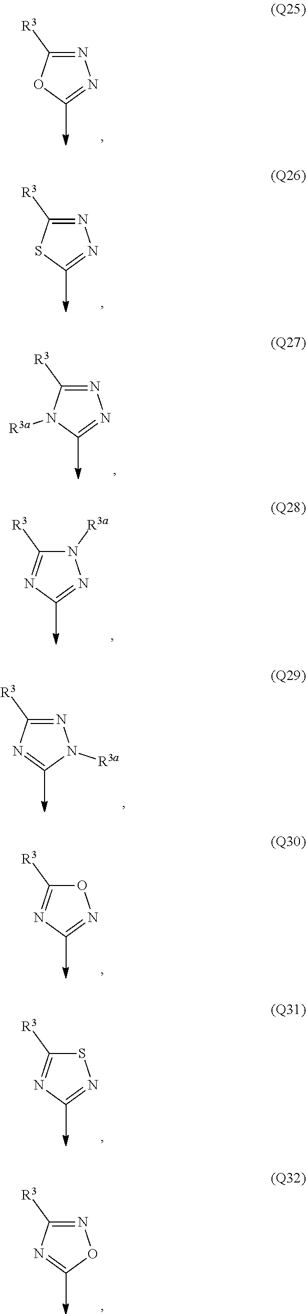 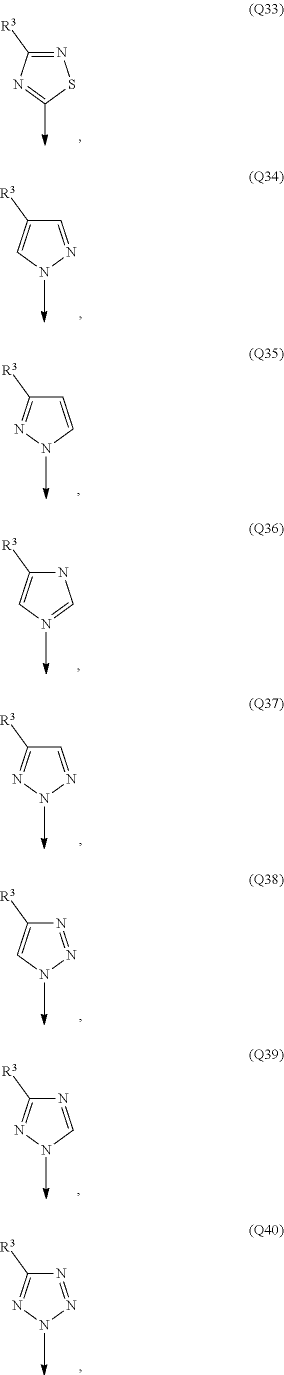 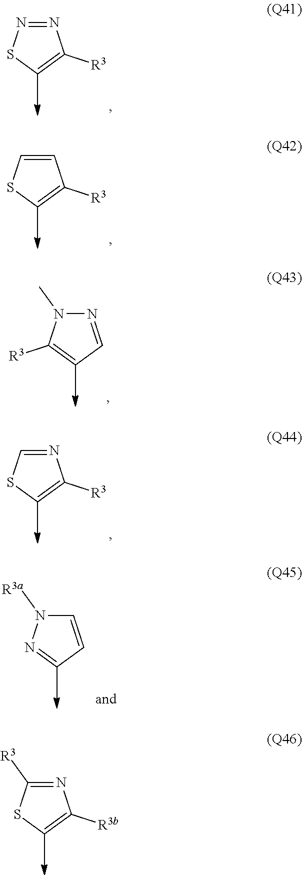
wherein R³ is hydrogen or as defined in claim 1 above, R^{3a} is selected from the group consisting of hydrogen, methyl, ethyl, CHF₂ and cyclopropyl and R^{3b} is selected from the group consisting of hydrogen, fluoro, chloro and bromo.

7. A compound according to claim 1, wherein Q is selected from the group consisting of Q-1, Q-2, Q-3, Q-4, Q-13, Q-14, Q-15, Q-16, Q-22, Q-23, Q-24, Q-41, Q-42 and Q-43.

8. A compound according to claim 1, wherein Q is selected from the group consisting of Q-1, Q-14, Q-22, Q-23, Q-41 and Q-43.

9. A compound according to claim 8, wherein R³ is chloro.

10. A herbicidal composition comprising a compound according to claim 1 and an agriculturally acceptable formulation adjuvant.

11. A herbicidal composition according to claim 10, further comprising at least one additional pesticide.

12. A herbicidal composition according to claim 11, wherein the additional pesticide is a herbicide or herbicide safener.

13. A method of controlling weeds at a locus comprising application to the locus of a weed controlling amount of a composition claim 10.

14. Use of a compound of Formula (I) as defined in claim 1 as a herbicide.

15. A compound of Formula (VIIIa): 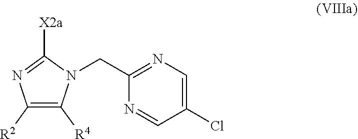
wherein R² and R⁴ are as defined in claim 1 above and X2a is hydrogen or halogen.
